(19) United States
(12) Patent Application Publication
(10) Pub. No.: US 2026/0138050 A1
(43) Pub. Date: May 21, 2026

(54) MATERIAL COMPOSITION OPTIMIZATION FOR PEROVSKITE DEVICES

(71) Applicant: NANYANG TECHNOLOGICAL UNIVERSITY, Singapore (SG)

(72) Inventors: Nripan MATHEWS, Singapore (SG); Kedar HIPPALGAONKAR, Singapore (SG); Emha Bayu MIFTAHULLATIF, Singapore (SG); Shreyas Dinesh PETHE, Singapore (SG)

(21) Appl. No.: 19/390,489

(22) Filed: Nov. 15, 2025

(30) Foreign Application Priority Data
Nov. 15, 2024 (SG) 10202403565R

(51) Int. Cl. B01D 9/00; G01N 27/12; G01N 33/00

(52) U.S. Cl. CPC B01D 9/0063; G01N 27/125; G01N 33/39; B01D 2009/0086

(57) ABSTRACT

A method of optimizing material compositions for perovskite devices is provided. The method includes, for each iteration of a plurality of iterations: providing a substrate having an array of multilayer device stacks formed thereon; infiltrating, using an automated liquid handling robot, each multilayer device stack of the array of multilayer device stacks formed on the substrate associated with the iteration with a perovskite solution having a selected precursor composition from a set of different precursor compositions associated with the iteration to form a corresponding perovskite device, resulting in an array of perovskite devices associated with the iteration; performing, using an automated electrical measurement probing robot, electrical characterization on each perovskite device of the array of perovskite devices for generating electrical characterization data associated with the array of perovskite devices associated with the iteration; and predicting, using an AI model, a new set of different precursor compositions for a next iteration or that the set of different precursor compositions associated with the iteration is optimal based on the electrical characterization data of the array of perovskite devices associated with the iteration. There is also provided a corresponding system for optimizing material compositions for perovskite devices.

### CROSS-REFERENCE TO RELATED APPLICATION

This application claims the benefit of priority of Singapore Patent Application No. 10202403565R filed on Nov. 15, 2024, the content of which being hereby incorporated by reference in its entirety for all purposes.

### TECHNICAL FIELD

The present invention generally relates to a method and a system for optimizing material compositions for perovskite devices.

### BACKGROUND

High-throughput experimentation as a new approach to accelerate materials development would be of great value to the field of metal halide perovskites. Metal halide perovskites have attracted substantial scientific interest over the past decade, particularly in photovoltaic (PV) applications, owing to their exceptional properties. They may be employed for various device applications, such as photovoltaic, LED, photo-detectors and neuromorphic devices. These ionic semiconductors are generally described by the ABX₃ structure, where A is a monovalent cation such as methylammonium (e.g., CH₃NH₃⁺ or MA⁺), formamidinium (e.g., CH(NH₂)₂⁺ or FA⁺) and inorganic cesium (e.g., Cs⁺); B is a divalent metal cation such as Pb²⁺, Sn²⁺, and Ge²⁺, and X is a halide-ion such as I⁻, Br⁻, and Cl⁻. For example, even considering three species per site at 10% molar increments, results in 287,496 possible compositional variations. The exploratory materials space is further expanded if one considers perovskite-inspired halide compositions, such as vacancy-ordered perovskites and double perovskites. Furthermore, a myriad choice of additives, solvents, and processing conditions play a critical role in determining device performance. Despite such frenetic activity, the exact mechanisms behind the improvement of these approaches as well as whether such diverse approaches can be combined to yield efficient and stable devices remain open questions. For example, previous studies that aim to identify novel perovskite compositions via high-throughput approaches have primarily focused on individual material properties, such as bandgap through rapid optical or hyperspectral imaging and extensive crystalline structure investigation of thin films.

Accordingly, the abundance of compositional variations in metal halide perovskite presents both an opportunity and an additional challenge. On the one hand, the large compositional space offers an opportunity to enhance the exceptional optoelectronic properties of these materials and mitigate the existing issues. However, the large compositional space also poses a significant challenge, as a systematic and strategic approach is required to effectively explore this vast compositional landscape and unlock the full potential of perovskite materials.

A need therefore exists to provide a method of optimizing material compositions for perovskite devices, as well as a system thereof, that seeks to overcome, or at least ameliorate, one or more deficiencies in conventional methods of optimizing material compositions for perovskite devices, and more particularly, with a high throughput along with enhanced device performance(s) (e.g., device efficiency and stability). It is against this background that the present invention has been developed.

### SUMMARY

According to a first aspect of the present invention, there is provided a method of optimizing material compositions for perovskite devices, the method comprising, for each iteration of a plurality of iterations:
providing a substrate having an array of multilayer device stacks formed thereon;
infiltrating, using an automated liquid handling robot, each multilayer device stack of the array of multilayer device stacks formed on the substrate associated with the iteration with a perovskite solution having a selected precursor composition from a set of different precursor compositions associated with the iteration to form a corresponding perovskite device, resulting in an array of perovskite devices associated with the iteration;
performing, using an automated electrical measurement probing robot, electrical characterization on each perovskite device of the array of perovskite devices for generating electrical characterization data associated with the array of perovskite devices associated with the iteration; and
predicting, using an artificial intelligence (AI) model, a new set of different precursor compositions for a next iteration or that the set of different precursor compositions associated with the iteration is optimal based on the electrical characterization data of the array of perovskite devices associated with the iteration.

According to a second aspect of the present invention, there is provided a system for optimizing material compositions for perovskite devices, the system comprising:
an automated liquid handling robot configured to infiltrate each multilayer device stack of an array of multilayer device stacks formed on a substrate with a perovskite solution having a selected precursor composition from a set of different precursor compositions to form a corresponding perovskite device;
an automated electrical measurement probing robot configured to perform electrical characterization on each perovskite device of the array of perovskite devices for generating electrical characterization data associated with the array of perovskite devices; and
a computing system comprising:
   at least one memory; and
   at least one processor communicatively coupled to the at least one memory, the automated liquid handling robot and the automated electrical measurement probing robot and configured to, for each iteration of a plurality of iterations:
set the automated liquid handling robot with precursor composition set information on a set of different precursor compositions associated with the iteration for the automated liquid handling robot to infiltrate each multilayer device stack of an array of multilayer device stacks formed on a substrate associated with the iteration with a perovskite solution having a selected precursor composition from the set of different precursor compositions associated with the iteration to form a corresponding perovskite device, resulting in an array of perovskite devices associated with the iteration;
control the automated electrical measurement to perform electrical characterization on each perovskite device of the array of perovskite devices for generating electrical characterization data associated with the array of perovskite devices associated with the iteration; and
predict, using an AI model, a new set of different precursor compositions for a next iteration or that the set of different precursor compositions associated with the iteration is optimal based on the electrical characterization data of the array of perovskite devices associated with the iteration.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic diagram of a method of optimizing material compositions for perovskite devices, according to various embodiments of the present invention;

FIG. 2 depicts a system for optimizing material compositions for perovskite devices, according to various embodiments of the present invention;

FIG. 3 depicts a schematic diagram illustrating an overview of an example high-throughput (HT) method of optimizing material compositions for halide perovskite devices, according to various example embodiments of the present invention;

FIG. 4A depicts another schematic diagram showing an overview of the method of optimizing material compositions for halide perovskite devices, according to various example embodiments of the present invention;

FIG. 4B shows box plots of a five-loop power conversion efficiency (PCE) measurements associated with the method of optimizing material compositions according to various example embodiments of the present invention, compared with manual methods;

FIG. 4C depicts a bar diagram showing a comparison of fabrication times for 648 devices using manual spin coating compared with the method of optimizing material compositions according to various example embodiments of the present invention;

FIG. 4D depicts a bar diagram showing a comparison of J-V characterization times for 648 devices with manual methods compared with the method of optimizing material compositions according to various example embodiments of the present invention;

FIG. 5A shows box plots of V_{OC}, J_{SC}, FF, and PCE for different compositions (different perovskite source precursors (MAPbI₃/AVAI, MAPbI₃/MACl, and pristine MAPbI₃) of different mixing ratios) in Batch 1;

FIG. 5B shows median values in ternary space, with an interpolated heatmap showing comprehensive visualization of performance mapping across the mixing space;

FIG. 5C depicts line graphs showing device stability tests over 210 hours, which reveals unique V_{OC}, J_{SC}, FF, and PCE evolution for each composition, underscoring distinct stability behaviors;

FIG. 5D shows Table 1 presenting the precursor compositions for Batch 1 experiment, including 21 compositions with varying volume ratios of MAPbI₃/AVAI:MAPbI₃/MACl:MAPbI₃;

FIG. 6A depicts plots showing true (measured) vs predicted values of the GP Regressor trained with Peak PCE and ΔPCE from Batch 1 (darker shaded dots) and predictions for six new candidates (lighter shaded dots);

FIG. 6B shows the peak PCE and ΔPCE for actual measurements of Batch 1 and predicted Peak PCE and ΔPCE for Bayesian Optimization (BO) candidates;

FIG. 6C shows Table 2 presenting precursor composition candidates predicted or recommended by Bayesian Optimization for Batch 2;

FIGS. 6D to 6F show the performance of Batch 2, including the PCE evolution over 210 hours (FIG. 6D); the Peak PCE (FIG. 6E) and the −ΔPCE (FIG. 6F) per composition;

FIG. 6G shows experimental results plotted in Peak PCE vs ΔPCE space which shows Batch 2 notably expanding the Peak PCE×ΔPCE hypervolume relative to Ref_pt;

FIG. 6H shows cumulative hypervolume (HV) (Peak PCE×ΔPCE relative to Ref_pt) for cells in both batches highlights Composition 2 in Batch 2 as the champion;

FIG. 6I illustrates the HV gradation across the ternary space, indicating that 20-40% MAPbI3/AVAI yields strong performance;

FIGS. 6J and 6K show the final models for normalized Peak PCE and ΔPCE, respectively;

FIGS. 6L to 6O show BO recommendation for Batch 3 and their predicted achievements (Peak PCE and −(Peak−Final PCE), in several trials;

FIGS. 7A and 7B show V_{OC} versus ln(Light intensity) plots (Light intensity dependence V_{OC} measurement result) for Batches 1 and 2, respectively, from which n_{ID} was extracted;

FIG. 8A illustrates a ternary plot of median n_{ID} values, revealing lower n_{ID} in regions with low MAPbI₃/AVAI ratios;

FIG. 8B shows J-V characteristics of selected samples, including a champion device (AVAI+MACl), AVAI-only, MACl-only, and pristine devices at 210 hours;

FIG. 8C shows the comparative Incident Photon-to-Current Efficiency (IPCE), which shows AVAI enhances short-wavelength IPCE, while MACl improves long-wavelength IPCE;

FIG. 8D shows digital photographs of cells after nine months, where visible PbI₂ degradation product (yellow areas) is minimal in the champion device;

FIG. 8E shows the 2θ-XRD patterns of four representative samples (measured from the ZrO₂ side after carbon removal, postfabrication);

FIG. 8F shows grain size of perovskite crystals in the selected samples calculated from the XRD peak after Rietveld refinement;

FIG. 9 shows the intensity ratio of (110) peak as the dominant peak compares to other non-dominant peaks of selected samples;

FIGS. 10A to 10D show the cross-sectional SEM image of (a) pristine sample, (b) MACl only, (c) AVAI only, and (d) Champion sample, respectively;

FIG. 11 shows the 2θ-XRD patterns of planar samples after removal from glovebox (0 h) and after 24 hours of exposure in ambient air (24 h);

FIG. 12 shows Table 3 presenting example screen parameters of different layers for the mesoscopic device, according to various example embodiments of the present invention;

FIGS. 13A to 13D illustrate scaffold thickness homogeneity, including the boxed squares show the cell positions that undergo thickness measurements (FIG. 13A), and thickness measurement results of m-TiO₂, ZrO₂, and Carbon layer, respectively (FIGS. 13B to 13D);

FIG. 13E shows Table 4 presenting the thickness measurement results of m-TiO₂, ZrO₂, and Carbon layers;

FIGS. 14A to 14D show precursors volume optimization for infiltration, including scaffold cell infiltration with and without Kapton-tape as a confinement, with different volumes (FIG. 14A); the area coverage of perovskite from the back side (glass) of cells with Kapton tape and without Kapton tape respectively (FIGS. 14B and 14C); the plot of Area coverage (%) against drop cast volume (FIG. 14D);

FIGS. 15A to 15F illustrate scaffold resistance homogeneity, including scaffold resistance of Batch 1 (1), Batch 1 (2), and Batch 2 (1) according to the cell's position on the substrates (FIGS. 15A to 15C); and the scaffold resistance according to cell numbers counted from cell A1 to I9 (FIGS. 15D to 15F);

FIGS. 16A to 16D show the heatmaps of perovskite solar cell performance parameters based on the cell position for V_{OC} (FIG. 16A), J_{SC} (FIG. 16B), FF (FIG. 16C) and PCE (FIG. 16D;

FIGS. 16E to 16H show plots of the perovskite solar cell performance parameters' values against Scaffold R;

FIGS. 17A to 17E show two-probe vs four-probe measurement of (a) V_{OC}, (b) J_{SC}, (c) FF, (d) PCE, and (e) Rₛₑᵣᵢₑₛ, respectively, extracted from J-V characteristics;

FIGS. 18A to 18D show solar cell performance parameters versus the mask size for (a) V_{OC}, (b) J_{SC}, (c) FF, and (d) PCE, respectively;

FIGS. 19A to 19D present results showing the effect of measurement sequence on PV parameters;

FIGS. 20A to 20D show perovskite solar cell performance parameters acquired from the J-V curves in continuous measurement for (a) V_{OC}, (b) J_{SC}, (c) FF, and (d) PCE;

FIG. 21 shows Table 5 presenting the data spread extracted from boxplot of perovskite solar cell parameters acquired by this platform and manual measurement from reference; and

FIGS. 22A and 22B show Tables 6 and 7 presenting device fabrication speed and device characterization speed comparisons between the HT platform, according to various example embodiments of the present invention and conventional manual work.

### DETAILED DESCRIPTION

Various embodiments of the present invention provide a method and a system for optimizing material compositions for perovskite devices, and more particularly, halide perovskite devices.

As discussed in the background, high-throughput experimentation as a new approach to accelerate materials development would be of great value to the field of metal halide perovskites in view of the extremely large number of possible compositional variations (exploratory materials space) of metal halide perovskites. However, whether there can be a high-throughput approach that yields devices with enhanced performance(s) (e.g., efficient and stable devices) still remain an open question. In this regard, various embodiments of the present invention provide a method of optimizing material compositions for perovskite devices, as well as a system thereof, that seeks to overcome, or at least ameliorate, one or more deficiencies in conventional methods of optimizing material compositions for perovskite devices, and more particularly, with a high throughput along with enhanced device performance(s) (e.g., device efficiency and stability).

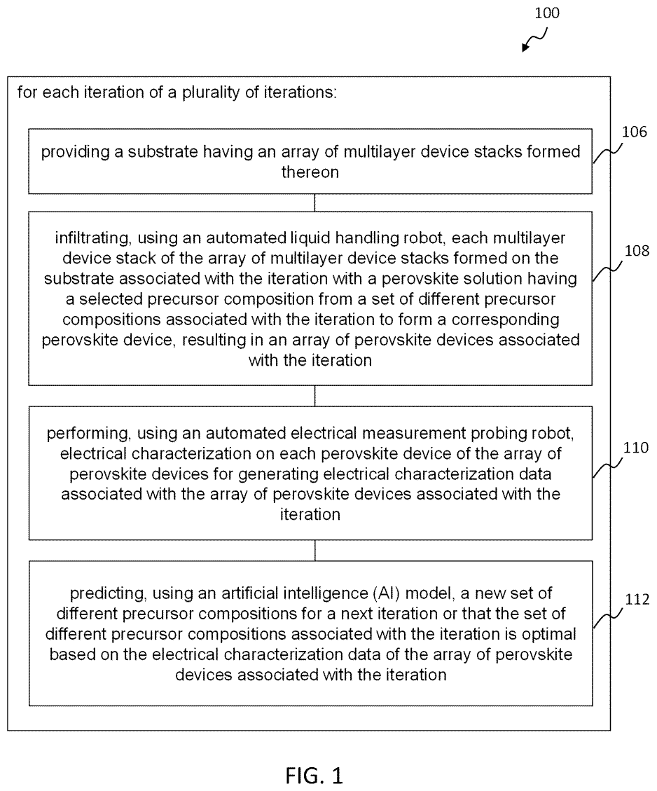

FIG. 1 depicts a schematic diagram of a method 100 of optimizing material compositions for perovskite devices, according to various embodiments of the present invention. The method 100 comprises, for each iteration of a plurality of iterations (optimization iterations): providing (at 106) a substrate having an array of multilayer device stacks formed thereon; infiltrating (at 108), using an automated liquid handling robot, each multilayer device stack of the array of multilayer device stacks formed on the substrate associated with the iteration with a perovskite solution having a selected precursor composition from (selected from) a set of different precursor compositions associated with the iteration to form a corresponding perovskite device, resulting in an array of perovskite devices associated with the iteration; performing (at 110), using an automated electrical measurement probing robot, electrical characterization on each perovskite device of the array of perovskite devices for generating electrical characterization data associated with the array of perovskite devices associated with the iteration (e.g., including electrical characterization data of each perovskite device of the array of perovskite devices associated with the iteration may be generated); and predicting (at 112), using an artificial intelligence (AI) model, a new set of different precursor compositions for a next iteration or that the set of different precursor compositions associated with the iteration is optimal based on the electrical characterization data of the array of perovskite devices associated with the iteration (for optimizing material compositions for perovskite devices). In various embodiments, the electrical characterization data may be obtained over a period of time, such as at each predefined interval.

In various embodiments, for each iteration of the plurality of iterations, each perovskite device of the array of perovskite devices associated with the iteration is a full-stack perovskite device.

In various embodiments, for each iteration of the plurality of iterations, each multilayer device stack of the array of multilayer device stacks is screen printed on the substrate. In this regard, each layer of the multilayer device stack is screen printed layer-by-layer over the substrate.

In various embodiments, for each iteration of the plurality of iterations, each multilayer device stack of the array of multilayer device stacks comprises a plurality of mesoporous layers. Therefore, each multilayer device stack is a mesoporous or mesoscopic multilayer device stack.

In various embodiments, the method 100 further comprises, for each iteration of the plurality of iterations: preparing, using the automated liquid handling robot and for each multilayer device stack of the array of multilayer device stacks associated with the iteration, the perovskite solution having the selected precursor composition for the multilayer device stack, including automated precursor mixing to obtain the perovskite solution having the selected precursor composition for infiltrating the multilayer device stack. In this regard, in various embodiments, based on precursor composition set information on a set of different precursor compositions associated with the iteration (e.g., predicted for the iteration), the automated liquid handling robot may automatically, for each multilayer device stack of the array of multilayer device stacks associated with the iteration, mix different perovskite source precursors (e.g., “mother” solutions) provided to obtain the perovskite solution having the selected precursor composition (i.e., with the corresponding mixing ratio defined by the precursor composition set information) for infiltrating the multilayer device stack.

In various embodiments, the AI model is an optimization model configured to predict the new set of different precursor compositions for the next iteration or that the set of different precursor compositions associated with the iteration is optimal based on the electrical characterization data of each perovskite device of the array of perovskite devices associated with the iteration with respect to one or more performance objectives for said each perovskite device based on different precursor compositions comprising different perovskite source precursors of different mixing ratios associated with the iteration.

In various embodiments, the optimization model is a Bayesian Optimization (BO) model comprising one or more surrogate models (e.g., Gaussian Process (GP) surrogate models) and an acquisition function (e.g., qNEHVI acquisition function). In this regard, for each iteration of the plurality of iterations, the method 100 further comprises: training, for each of the one or more surrogate models, the surrogate model based on the electrical characterization data of each perovskite device of the array of perovskite devices associated with the iteration with respect to a corresponding performance objective of the one or more performance objectives based on different precursor compositions comprising different perovskite source precursors of different mixing ratios associated with the iteration (i.e., mixing ratios as input to each surrogate model); and predicting, using the acquisition function, the new set of different precursor compositions for the next iteration or that the set of different precursor compositions associated with the iteration is optimal based on the one or more surrogate models. Accordingly, in various embodiments, a respective surrogate model is trained for each performance objective.

In various embodiments, the method 100 may be performed iteratively, such as until a predefined condition is met (e.g., when the new set of different precursor compositions predicted at a current iteration is predicted or determined to be optimal (e.g., the set of different precursor compositions predicted for a next iteration is predicted to not result in device performance improvements or worsen device performance).

In various embodiments, for each iteration of the plurality of iterations, the array of multilayer device stacks formed on the substrate is an array of solar cells, and each solar cell of the array of solar cells comprises an electron transport layer, a spacer layer and a hole transport layer. In various embodiments, the electron transport layer comprises a mesoporous TiO₂ layer, the spacer layer comprises a mesoporous ZrO₂ layer and the hole transport layer comprises a mesoporous carbon layer.

In various embodiments, for each iteration of the plurality of iterations, the set of different precursor compositions comprises different additive compositions.

In various embodiments, for each iteration of the plurality of iterations, the electrical characterization data associated with the array of perovskite devices associated with the iteration comprises one or more of open-circuit voltage measurement data, short-circuit current density measurement data, fill factor measurement data and power conversion efficiency (PCE) measurement data. In various embodiments, each of the one or more of open-circuit voltage measurement data, short-circuit current density measurement data, fill factor measurement data and power conversion efficiency (PCE) measurement data may be obtained over a period of time, such as at each predefined interval, and may thus be referred to as evolution data.

In various embodiments, for each iteration of the plurality of iterations, the electrical characterization on each perovskite device of the array of perovskite devices is performed using the automated electrical measurement probing robot according to a four-probe measurement configuration comprising two probes configured to source and sink current through the perovskite device and two probes configured to sense a potential difference across the perovskite device such that current and voltage paths are separated.

In various embodiments, for each iteration of the plurality of iterations, the electrical characterization on each perovskite device of the array of perovskite devices (perovskite solar cells) is performed further using an automated translation stage configured to move the substrate having the array of perovskite devices associated with the iteration supported thereon (e.g., for performing the electrical characterization on each perovskite device individually (i.e., one by one)) and a solar simulator configured to illuminate light that simulates sunlight onto the perovskite device. In various embodiments, a shadow mask comprising an array of holes is arranged to align with the array of solar cells (i.e., the array of holes aligned with (to oppose) the array of solar cells). In this regard, the solar simulator is configured to illuminate light that simulates sunlight onto the perovskite device through the shadow mask.

In various embodiments, the optimization model is a multi-objective optimization model. In various embodiments, the one or more performance objectives comprise a peak power conversion efficiency (peak PCE) objective defined to maximize a peak power conversion efficiency of perovskite devices (e.g., perovskite solar cells) and a power conversion efficiency change (ΔPCE) objective defined to minimize a power conversion efficiency change of perovskite devices (e.g., perovskite solar cells).

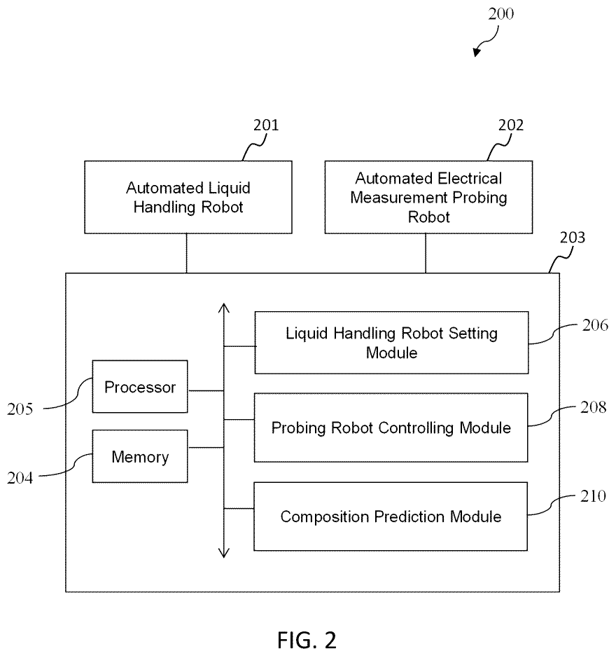

FIG. 2 depicts a system 200 for optimizing material compositions for perovskite devices, according to various embodiments of the present invention, corresponding to the method 100 of optimizing material compositions for perovskite devices as described hereinbefore with reference to FIG. 1 according to various embodiments of the present invention. The system 200 comprises: an automated liquid handling robot 201 configured to infiltrate each multilayer device stack of an array of multilayer device stacks formed on a substrate with a perovskite solution having a selected precursor composition from a set of different precursor compositions to form a corresponding perovskite device; an automated electrical measurement probing robot 202 configured to perform electrical characterization on each perovskite device of the array of perovskite devices for generating electrical characterization data associated with the array of perovskite devices; and at least one computing system 203. The computing system 203 comprises: at least one memory 204; and at least one processor 205 communicatively coupled to the at least one memory 204, the automated liquid handling robot 201 and the automated electrical measurement probing robot 202 and configured to, for each iteration of a plurality of iterations: set the automated liquid handling robot 201 with precursor composition set information on a set of different precursor compositions associated with the iteration (e.g., by sending/communicating the precursor composition set information or data to the automated liquid handling robot 201 based on wired or wireless communication) for the automated liquid handling robot to infiltrate each multilayer device stack of an array of multilayer device stacks formed on a substrate associated with the iteration with a perovskite solution having a selected precursor composition from the set of different precursor compositions associated with the iteration to form a corresponding perovskite device, resulting in an array of perovskite devices associated with the iteration; control the automated electrical measurement probing robot 202 to perform electrical characterization on each perovskite device of the array of perovskite devices for generating electrical characterization data associated with the array of perovskite devices associated with the iteration; and predict, using an AI model, a new set of different precursor compositions for a next iteration or that the set of different precursor compositions associated with the iteration is optimal based on the electrical characterization data of the array of perovskite devices associated with the iteration (for optimizing material compositions for perovskite devices).

It will be appreciated by a person skilled in the art that the automated liquid handling robot 201 is not limited to any specific type or configuration as long as the automated liquid handling robot 201 is configured to be operable (e.g., controllable based on instructions) to infiltrate each multilayer device stack of an array of multilayer device stacks formed on a substrate with a perovskite solution having a selected precursor composition from a set of different precursor compositions, and in various embodiments, as long as the automated liquid handling robot 201 is further configured to be operable to prepare, for each multilayer device stack of the array of multilayer device stacks, the perovskite solution having the selected precursor composition for the multilayer device stack, including automatic precursor mixing to obtain the perovskite solution having the selected precursor composition for infiltrating the multilayer device stack. Furthermore, various types of liquid handling robots are known in the art and thus need not be described herein for clarity and conciseness. Similarly, it will be appreciated by a person skilled in the art that the automated electrical measurement probing robot 202 is not limited to any specific type or configuration as long as the automated electrical measurement probing robot 202 is configured to be operable to perform electrical characterization on each perovskite device of the array of perovskite devices for generating electrical characterization data associated with the array of perovskite devices.

In various embodiments, the system 200 further comprises: an automated translation stage configured to, for each iteration of the plurality of iterations, move the substrate having the array of perovskite devices associated with the iteration supported thereon; and a solar simulator configured to, for each iteration of the plurality of iterations, illuminate light that simulates sunlight onto the substrate associated with the iteration. In this regard, the at least one processor is further configured to, for each iteration of the plurality of iterations: control the automated translation stage to move the substrate having the array of perovskite devices associated with the iteration supported thereon for performing, for each perovskite device of the array of perovskite devices associated with the iteration, the electrical characterization on the perovskite device and for the solar simulator to illuminate the light onto the perovskite device. For example, the automated liquid handling robot 201 and the automated electrical measurement probing robot 202 may each be arranged or configured accordingly, together with various structures and mechanisms (e.g., support structures and linear positioning stages (e.g., XY translation stages)) as desired or as appropriate, in order to perform corresponding operations as described herein according to various embodiments of the present invention. In this regard, it will be appreciated by a person skilled in the art that the automated liquid handling robot 201 and the automated electrical measurement probing robot 202 are not limited to any specific arrangements or configurations, together with various structures and mechanisms, as long as the automated liquid handling robot 201 and the automated electrical measurement probing robot 202 are able to perform corresponding operations as described herein according to various embodiments of the present invention.

In various embodiments, the system 200 may further comprise a perovskite device array forming apparatus configured to form, for each iteration of the plurality of iterations, the array of perovskite devices on the substrate associated with the iteration. In various embodiments, the perovskite device array forming apparatus may be a screen printing apparatus for forming the array of multilayer device stacks on the substrate via screen printing. In this regard, each layer of each multilayer device stack is screen printed layer-by-layer over the substrate.

It will be appreciated by a person skilled in the art that the at least one processor 205 may be configured to perform various functions or operations through set(s) of instructions (e.g., software modules) executable by the at least one processor 205 to perform various functions or operations. Accordingly, as shown in FIG. 2, the computing system 203 may comprise: a liquid handling robot setting module (or a liquid handling robot setting circuit) 206 configured to set the automated liquid handling robot 201 with precursor composition set information on a set of different precursor compositions associated with the iteration (e.g., by sending/communicating the precursor composition set information or data to the automated liquid handling robot 201 based on wired or wireless communication) for the automated liquid handling robot 201 to infiltrate each multilayer device stack of an array of multilayer device stacks formed on a substrate associated with the iteration with a perovskite solution having a selected precursor composition from the set of different precursor compositions associated with the iteration to form a corresponding perovskite device, resulting in an array of perovskite devices associated with the iteration; a probing robot controlling module (or a probing robot controlling circuit) 208 configured to control the automated electrical measurement probing robot 202 to perform electrical characterization on each perovskite device of the array of perovskite devices for generating electrical characterization data associated with the array of perovskite devices associated with the iteration; and a composition prediction module (or a composition prediction circuit) 210 configured to predict, using an AI model, a new set of different precursor compositions for a next iteration or that the set of different precursor compositions associated with the iteration is optimal based on the electrical characterization data of the array of perovskite devices associated with the iteration (for optimizing material compositions for perovskite devices).

In various embodiments, as described hereinbefore with respect to the method 100, the computing system 203 may correspondingly be configured to perform the material composition optimization iteratively, such as until a predefined condition is met (e.g., when the new set of different precursor compositions predicted at a current iteration is predicted or determined to be optimal (e.g., the set of different precursor compositions predicted for a next iteration is predicted to not result in device performance improvements or worsen device performance).

It will be appreciated by a person skilled in the art that the above-mentioned modules of the computing system 203 are not necessarily separate modules, and two or more modules may be realized by or implemented as one functional module (e.g., a circuit or a software program) as desired or as appropriate without deviating from the scope of the present invention. For example, two or more of the liquid handling robot setting module 206, the probing robot controlling module 208 and the composition prediction module 210 may be realized (e.g., compiled together) as one executable software program (e.g., software application), which for example may be stored in the at least one memory 204 and executable by the at least one processor 205 to perform the corresponding functions or operations as described herein according to various embodiments of the present invention.

In various embodiments, the computing system 203 is configured for optimizing material compositions for perovskite devices and corresponds to the method 100 for optimizing material compositions for perovskite devices as described hereinbefore with reference to FIG. 1, therefore, various operations, functions or steps configured to be performed by the at least one processor 205 may correspond to various operations, functions or steps of the method 100 of optimizing material compositions as described hereinbefore according to various embodiments, and thus need not be repeated with respect to the computing system 203 (or the system 200) for optimizing material compositions for clarity and conciseness. In other words, various embodiments described herein in context of methods (e.g., the method 100 of optimizing material compositions for perovskite devices) are analogously valid for the corresponding systems or devices (e.g., the computing system 203 (or the system 200) for optimizing material compositions for perovskite devices), and vice versa. For example, in various embodiments, the at least one memory 204 may have stored therein the liquid handling robot setting module 206, the probing robot controlling module 208 and/or the composition prediction module 210, which respectively correspond to various operations, functions or steps of the method 100 of optimizing material compositions as described hereinbefore according to various embodiments, which are executable by the at least one processor 205 to perform the corresponding operations, functions or steps as described herein.

A computing system, a controller, a microcontroller or any other system providing a processing capability may be provided according to various embodiments in the present invention. Such a system may be taken to include one or more processors and one or more computer-readable storage mediums. For example, the computing system 203 for optimizing material compositions for perovskite devices described hereinbefore may include at least one processor (or controller) and at least one computer-readable storage medium (or memory) which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory). Furthermore, it will be appreciated by a person skilled in the art that the system 203 for optimizing material compositions may be implemented by a high-performance computer system, or a network of high-performance computer systems, known in the art for performing training, especially when a large-scale training is performed. For example, the high-performance computer system may include multiple processors including GPUs (graphics processing units) and CPUs (central processing units) optimized for advanced computing tasks, such as execution of machine learning algorithms. For example, the high-performance computer system may comprise an array of GPUs (Graphics Processing Units) dedicated to handling parallel processing tasks to facilitate the rapid execution of machine learning algorithms. In the field of machine learning, it will be understood by a person skilled in the art that the system 203 for optimizing material compositions may be implemented as a server (e.g., centralized or distributed server (centralized or decentralized machine learning)), such as a cloud server, including specialized hardware (e.g., high performance graphics processing unit(s) (GPU(s))) designed for handling resource-intensive tasks associated with machine learning.

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of various functions or operations may also be understood as a “circuit” in accordance with various other embodiments. Similarly, a “module” may be a portion of a system according to various embodiments in the present invention and may encompass a “circuit” as above, or may be understood to be any kind of a logic-implementing entity therefrom.

Some portions of the present disclosure may be explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm may be, and generally, conceived to be a self-consistent sequence of steps leading to a desired result.

The present specification also discloses a computing system (e.g., which may also be embodied as one or more devices or apparatuses), such as the computing system 203 for performing various operations, functions or steps of various methods described herein. Such computing systems may each be specially constructed for the required purposes or may comprise a general purpose computer system selectively activated or reconfigured by a computer program stored in the computer system. In general, various algorithms that may be presented herein are not limited to being implemented or executed by any particular computer system. Alternatively, the construction of more specialized computer system (e.g., a high-performance computer system as described hereinbefore) to perform various operations, functions or steps of various methods described herein may be provided as desired or as appropriate without going beyond the scope of the present invention.

In addition, the present specification also at least implicitly discloses computer program(s) or software/functional module(s), in that it would be apparent to a person skilled in the art that various operations, functions or steps of various methods described herein may be put into effect by computer code. The computer program(s) is not intended to be limited to any particular programming language and implementation thereof, and it will be appreciated by a person skilled in the art that a variety of programming languages and coding thereof may be used to implement the computer program(s). Moreover, the computer program(s) is not intended to be limited to any particular control flow as there are a variety of programming languages which can use different control flows. It will be appreciated by a person skilled in the art that a computer program may be stored on any computer-readable storage medium (non-transitory computer-readable storage medium), such as but not limited to, a magnetic disk, an optical disk or a memory chip. For example, a computer program stored on a computer-readable storage medium may be loaded and executed on a computer system to implement various operations, functions or steps of various methods described herein according to various embodiments of the present invention.

Accordingly, in various embodiments, there is provided a computer program product, embodied in one or more computer-readable storage mediums (non-transitory computer-readable storage medium), comprising instructions (e.g., the liquid handling robot setting module 206, the probing robot controlling module 208 and/or the composition prediction module 210) executable by one or more computer processors to perform various operations, functions or steps as described hereinbefore according to various embodiments of the present invention for optimizing material compositions for perovskite devices. Accordingly, various computer programs or software modules described herein may be stored in a computer program product receivable by a computing system therein, such as the computing system 203 configured for optimizing material compositions for perovskite devices as shown in FIG. 2, for execution by at least one processor 205 of the computing system 203 to perform various operations, functions or steps of various methods described herein according to various embodiments of the present invention.

It will be appreciated by a person skilled in the art that various modules of computing systems described herein (e.g., the liquid handling robot setting module 206, the probing robot controlling module 208 and/or the composition prediction module 210) may be software module(s) realized by computer program(s) or set(s) of instructions executable by a computer processor to perform various functions or operations. Various modules of computing systems described herein may also be implemented as hardware module(s) being functional hardware unit(s) designed to perform various functions or operations. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC) or a Field Programmable Gate Array (FPGA). Numerous other possibilities exist. It will also be appreciated by a person skilled in the art that a combination of hardware and software modules may be implemented. Furthermore, various operations, functions or steps of various methods described herein may be performed in parallel rather than sequentially as desired or as appropriate (e.g., as long as it does not render the method(s) inoperable or unsatisfactory for its intended purpose).

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Any reference to an element or a feature herein using a designation such as “first”, “second” and so forth does not limit the quantity or order of such elements or features, unless stated or the context requires otherwise. For example, such designations may be used herein as a convenient way of distinguishing between two or more elements or instances of an element. Thus, a reference to first and second elements does not necessarily mean that only two elements can be employed, or that the first element must precede the second element, unless stated or the context requires otherwise. In addition, a phrase referring to “at least one of” a list of items refers to any single item therein or any combination of two or more items therein.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

In particular, for better understanding of the present invention and without limitation or loss of generality, various example embodiments of the present invention will now be described with respect to a high-throughput method of optimizing material compositions for halide perovskite devices, and more specifically, perovskite solar cells, within MAPbI₃, MAPbI₃/AVAI, and MAPbI₃/MACl additive composition space with respect to device efficiency and stability (optimization objectives or device performance objectives). However, it will be understood by a person skilled in the art that the method of optimizing material compositions for perovskite device is not limited to such specific perovskite solar cells or such a specific composition space (or such a specific ternary composition space), and other electronic or optoelectronic devices, other composition spaces utilizing other material components (as well as of composition spaces with different number of material components (or dimensions)) may be provided as desired or as appropriate without going beyond the scope of the present invention. In other words, the method is not limited to any specific perovskite devices or any specific exploratory material composition space for perovskite devices. By way of examples and without limitations, other perovskite devices include perovskite memristor devices, perovskite-based neuromorphic devices, perovskite-based photoconductive devices, perovskite diodes (e.g., perovskite light emitting diodes (PeLEDs), perovskite photodetectors and so on. In this regard, using screen printing, a stack of porous mesoscopic layers can be fabricated that can serve as a versatile platform for various electronic or optoelectronic devices. In the case of perovskite solar cells, for example, TiO₂ is typically fabricated as the electron transport layer, ZrO₂ as the spacer layer, and carbon as both the hole transport layer and electrode. Such a multilayer device stack may then be infiltrated with a perovskite solution that functions as an active layer. In this regard, it will be appreciated by a person skilled in the art that other multilayer device stacks may be fabricated for other practical applications, such as the other example perovskite devices mentioned above. Furthermore, it will be appreciated by a person skilled in the art that the multilayer device stack may have various structures/architectures as well as various material layers as appropriate depending on the perovskite device types and functional requirements. In relation to the exploratory material composition space, in general, any combination of different perovskite source precursors (including any number of perovskite source precursors thereof) may employed as desired or as appropriate, such as but not limited to, based on the ABX₃ structure described in the background, Ruddlesden-Popper structure, Dion-Jacobson structure, Rudorffite structure and so on. It will also be understood by a person skilled in the art that the optimization objectives are not limited to device efficiency and stability, and other device performance objectives (as well as other number of device performance objectives) may be implemented as desired or as appropriate without going beyond the scope of the present invention, such as but not limited to, toxicity level and material price (for perovskite solar cells), on/off ratio, retention ratio, rectification ratio, gain, linearity/programmability (for perovskite memristor), and so on. Furthermore, the method of optimizing material compositions will be described based on multiobjective Bayesian Optimization (BO) as an example illustrative AI model. However, it will be understood by a person skilled in the art that the present invention is not limited to such a specific AI model and other types of AI models (or machine learning models), as well as various model architectures thereof, may be implemented as desired or as appropriate (e.g., depending on the optimization objective(s)), without going beyond the scope of the present invention, such as but not limited to, regression, classification or clustering machine learning models.

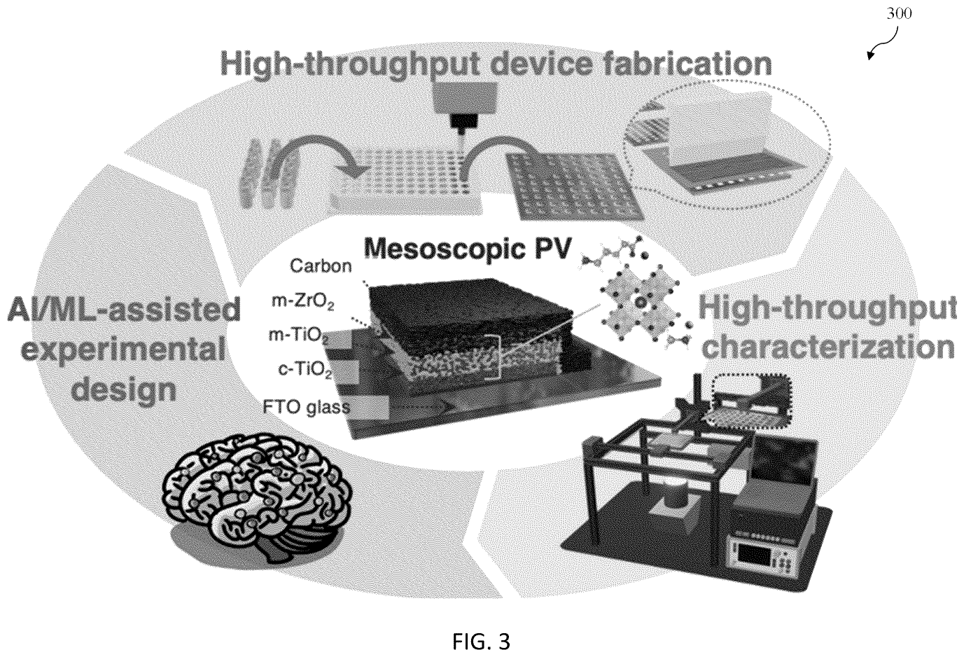

Metal halide perovskites offer a vast but largely unexplored compositional and processing space. High-throughput experimentation (HTE) integrated with machine learning (ML) is ideal for efficient exploration, preferably at the device level. However, multilayer deposition challenges often limit HTE to stand-alone materials. Various example embodiments advantageously address these challenges by, inter alia, employing a substrate with an array of solar cells screen-printed thereon (e.g., screen-printed triple-mesoscopic architecture), offering stability and low-cost fabrication, enabling rapid in-device screening of, for example, up to 81 unique devices per batch (per substrate). FIG. 3 depicts a schematic diagram illustrating an overview of an example high-throughput (HT) method 300 of optimizing material compositions for halide perovskite devices according to various example embodiments of the present invention. For example, the high-throughput method 300 (or the corresponding high-throughput platform) for optimizing material compositions for halide perovskite devices is found to be able to accelerate experimental throughput over 100× and reduces data variance to 25% of manual methods. As an illustrative example, the method 300 (based on a ML-driven workflow) may be configured or implemented to identify optimal additive compositions within MAPbI₃, MAPbI₃/AVAI, and MAPbI₃/MACl compositional space that simultaneously enhance device efficiency and stability. Prior additive studies were performed individually in conventional contexts, whereas the method 300, which adopts a HT/ML-assisted approach on full devices, according to various example embodiments of the present invention is unprecedented. As an illustrative example, the method 300 is found to achieve a 5.75-fold improvement over pristine MAPbI₃, validated across two experimental batches. Further analysis shows AVAI and MACl act synergistically—AVAI aids infiltration and early crystallization, while MACl suppresses long-term PbI₂ formation—together enhancing carrier dynamics and stability. Accordingly, various example embodiments advantageously provide a machine-learning-driven in-device optimization of all-printed perovskite solar cells.

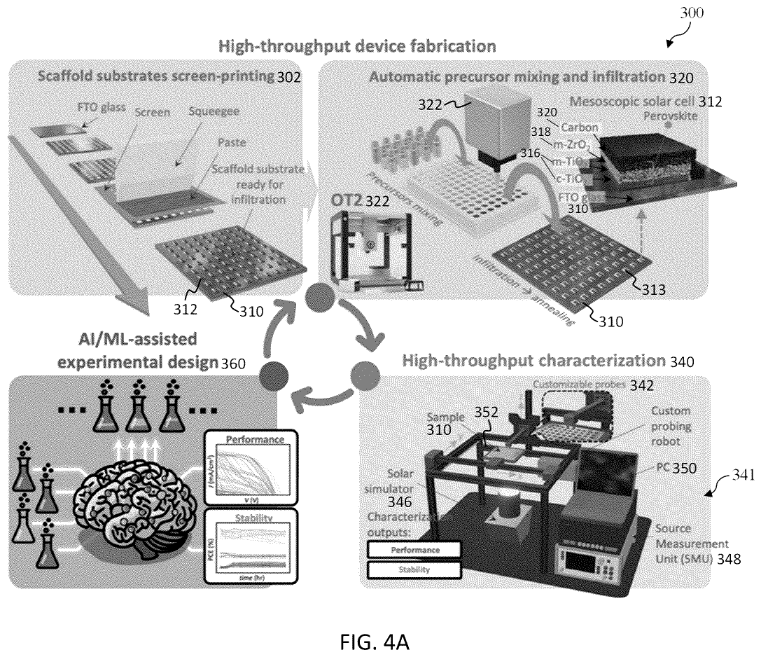

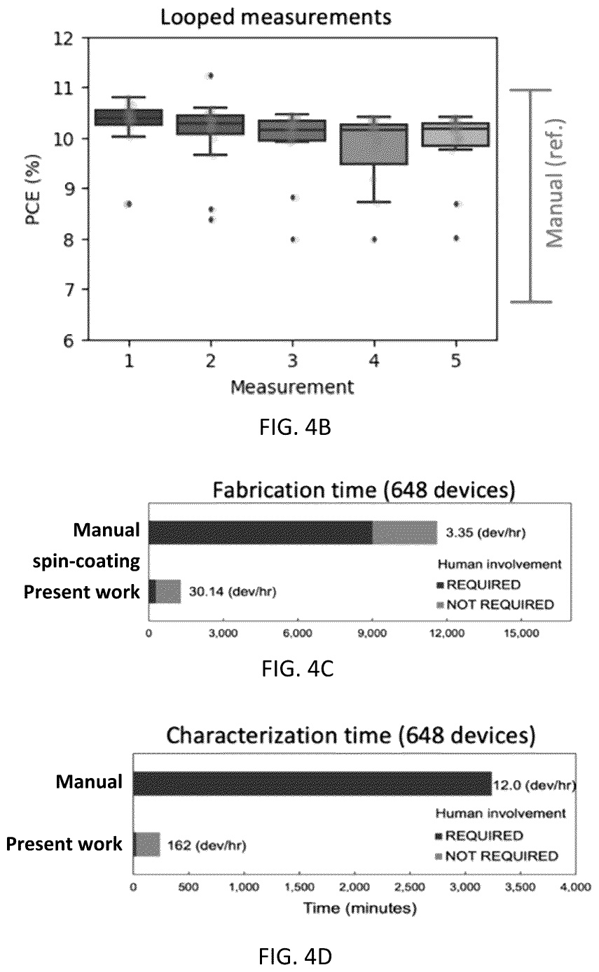

FIG. 4A depicts another schematic diagram showing an overview of the method 300 of optimizing material compositions for halide perovskite devices according to various example embodiments of the present invention, which also illustrates a corresponding example closed-loop, ML-driven HT platform for perovskite devices, integrating HT device fabrication (screen-printed substrate and automated liquid handling), HT characterization (automatic probing and characterization), and AI/MBL assisted experimental design. The method 300 comprises, for each iteration of a plurality of iterations: providing (e.g., forming as illustrated at 302) a substrate 310 having an array of solar cells 312 (multilayer device stacks) formed thereon; infiltrating (at 320), using an automated liquid handling robot 322, each solar cell of the array of solar cells 312 formed on the substrate 310 associated with the iteration with a perovskite solution having a selected precursor composition from a set of different precursor compositions associated with the iteration to form a corresponding perovskite solar cell 313, resulting in an array of perovskite solar cells 313 associated with the iteration; performing (at 340), using an automated electrical measurement probing robot 342, electrical characterization on each perovskite solar cell of the array of perovskite solar cells 313 for generating electrical characterization data associated with the array of perovskite solar cells 313 associated with the iteration; and predicting (at 360), using an artificial intelligence (AI) model, a new set of different precursor compositions for a next iteration or that the set of different precursor compositions associated with the iteration is optimal based on the electrical characterization data of the array of perovskite solar cells 313 associated with the iteration (for optimizing material compositions for halide perovskite devices), such as with respect to device efficiency and stability. For example, as shown in FIG. 4B, a five-loop PCE measurements show about 25% lower variance than manual methods. In addition, FIG. 4C depicts a bar diagram showing a comparison of fabrication times for 648 devices (8 scaffold substrates×81 cells per substrate) using manual spin coating vs the present method 300. Furthermore, FIG. 4D depicts a bar diagram showing a comparison of J-V characterization times for 648 devices with manual methods vs the present method 300.

Accordingly, as illustrated in FIG. 4A, various example embodiments develop a HT platform having a HT workflow which integrates device fabrication (at 302 and 320), characterization (at 340), and AI/ML-based experimental design (at 360) and, for example, utilizes directly measured photovoltaic efficiency and stability as performance objectives for active learning. In the HT device fabrication (at 302 and 320), according to various example embodiments of the present invention, a printable carbon-based mesoscopic solar cell structure is employed to overcome the challenge of rapidly fabricating full-stack devices. In this regard, various example embodiments note that the mesoscopic carbon-based architecture represents a widely adopted and practical configuration in perovskite photovoltaic research, development, and commercialization. Various example embodiments note that printable carbon-based perovskite solar cells are a promising design for commercial scaling, due to their low fabrication cost, high operational stability, and the absence of costly hole-transport layers. In various example embodiments, scaffold substrates may first be mass-produced using a screen-printing method. In various example embodiments, the screen-printed mesoscopic scaffold 312 (multilayer device stack) includes an electron transport layer (e.g., a compact/mesoporous TiO₂) 316, a spacer layer (e.g., mesoporous ZrO₂) 318, and a hole transport layer (e.g., mesoporous carbon) 320 formed on a substrate (e.g., FTO glass) 310. Accordingly, multiple solar cells 312 (e.g., the entire array of solar cells 312) can advantageously be fabricated at once on the substrate 310. This cell architecture is now considered a standard baseline for large-area, long-term stable perovskite solar cells, with several industrial initiatives actively advancing product development based on this design. In various example embodiments, this device configuration is advantageously adopted for directly exploring the effect of the additives and compositions on the device performance in a HT fashion and is directly relevant to the commercial trajectory of perovskite photovoltaic. Multiple scaffold substrates, for example, consisting of 81 cells per substrate, can be mass-produced. Accordingly, in various example embodiments, each solar cell (multilayer device stack) of the array of solar cells 312 is screen printed on the substrate 310, and furthermore, each solar cell of the array of solar cells 312 comprises a plurality of mesoporous layers.

Perovskite precursors with various compositions and additives (e.g., a set of different precursor compositions (e.g., comprising different additive compositions)) are automatically formulated using the liquid handling robot 322 and infiltrated into the scaffold cells (solar cells) 312, for example, producing up to 81 working perovskite solar cells 313 with unique active materials. Accordingly, for each iteration of the plurality of iterations (optimization iterations), the liquid handling robot 322 is operable or controllable (based on instructions) to prepare (e.g., based on precursor composition set information on a set of different precursor compositions associated with the iteration), for each solar cell of the array of solar cells 312 associated with the iteration, the perovskite solution having the selected precursor composition for the solar cell 312, including automated precursor mixing to obtain the perovskite solution having the selected precursor composition for the solar cell 312. For example, different perovskite source precursors may first be prepared manually in vials. The liquid handling robot 322 may then perform automated precursor mixing based on the different perovskite source precursors to obtain perovskite solutions (with different precursor compositions comprising different perovskite source precursors of different mixing ratios) in other vials/well plates. The hydrophobicity of the carbon layer 320 safeguards against moisture, enabling stable device fabrication under ambient conditions. Accordingly, the produced scaffold substrate is subsequently infiltrated with perovskite precursor solutions, which are automatically formulated (automated precursor mixing) using the liquid handling robot 322, followed by annealing to crystallize the perovskite. The perovskite crystals connect the electron transport layer and hole transport layer via the spacer. With this configuration, the perovskite deposition and subsequent annealing process are carried out as the final steps, ensuring that the target material under exploration remains unaffected by any other subsequent post-processing parameters. The hydrophobic nature of the carbon layers protects against moisture, which is a source of degradation in perovskite, thus enabling the fabrication of fully functional perovskite solar cells 313 in ambient atmospheric conditions with excellent device stability. Therefore, this example setup can fabricate up to 81 full-stack solar cells 312 on a single 100 cm² substrate, each solar cell 312 can be infiltrated with different precursor compositions.

In the HT characterization (at 340), an in-house robotic measurement system 341 comprising an automated electrical measurement probing robot 342, a solar simulator 346 (configured to illuminate light that simulates sunlight, e.g., a portable LED solar simulator) and a Source Measure Unit (SMU) 348 then performs fully automated electrical characterization. The automated electrical measurement probing robot 341 is configured to perform electrical characterization on each perovskite solar cell of the array of perovskite solar cells 313 for generating electrical characterization data associated with the array of perovskite solar cells 313. Accordingly, for each iteration of the plurality of iterations (optimization iterations), the automated electrical measurement probing robot 341 is operable or controllable to perform electrical characterization on each perovskite solar cell of the array of perovskite solar cells for generating electrical characterization data associated with the array of perovskite solar cells associated with the iteration.

In various example embodiments, the robotic measurement system 341 further comprises a computing system 350 comprising at least one processor communicatively coupled to the at least one memory, the automated liquid handling robot 322 and the automated electrical measurement probing robot 342 and configured to, for each iteration of a plurality of iterations: set the automated liquid handling robot 322 with precursor composition set information on a set of different precursor compositions associated with the iteration (e.g., by sending/communicating the precursor composition set information or data to the automated liquid handling robot 322 based on wired or wireless communication) for the automated liquid handling robot 322 to infiltrate each solar cell of an array of solar cells 312 formed on a substrate 310 associated with the iteration with a perovskite solution having a selected precursor composition from the set of different precursor compositions associated with the iteration to form a corresponding perovskite solar cell 313, resulting in an array of perovskite solar cells 313 associated with the iteration; and control the automated electrical measurement probing robot 342 to perform electrical characterization on each perovskite solar cell of the array of perovskite solar cells 313 for generating electrical characterization data associated with the array of perovskite solar cells 313 associated with the iteration. For example, the computing system 350 may be configured to communicate or interact with the automated liquid handling robot 322 and the automated electrical measurement probing robot 342, as well as the automated translation stage 352, based on Python API.

In various example embodiments, an automated translation stage 352 is provided and configured to, for each iteration of the plurality of iterations, move (e.g., XY translation) the substrate 310 having the array of perovskite solar cells 313 associated with the iteration supported thereon (i.e., using a substrate holder). In various a solar simulator configured to, for each iteration of the plurality of iterations, illuminate light that simulates sunlight onto the substrate 310 associated with the iteration. In this regard, the computing system 350 is further configured to, for each iteration of the plurality of iterations: control the automated translation stage 352 to move (e.g., XY translation) the substrate 310 having the array of perovskite solar cells 313 associated with the iteration supported thereon for performing, for each perovskite solar cell of the array of solar cells 313 associated with the iteration, the electrical characterization on the perovskite solar cell 313 and for the solar simulator 346 (e.g., fixed in position) to illuminate the light (e.g., effective light ray focused on small area (slightly bigger than 1 cell area)) onto the perovskite solar cell 313. Accordingly, in various example embodiments, for each iteration of the plurality of iterations, the computing system 350 is configured (e.g., based on programs or instructions therein) to control the movement of the substrate 310 (via the automated translation stage 352), the movement of the electrical measurement probing robot 342 (e.g., along the Z direction only), the solar simulator's on/off, the SMU measurement, as well as setting or providing the automated liquid handling robot 322 with the precursor composition set information.

Accordingly, in various example embodiments, fully automatic electrical characterization (e.g., J-V characterization) can be performed rapidly for the entire array of perovskite solar cells 313 (e.g., 81 perovskite solar cells) on each substrate 310 without or with very minimal human intervention.

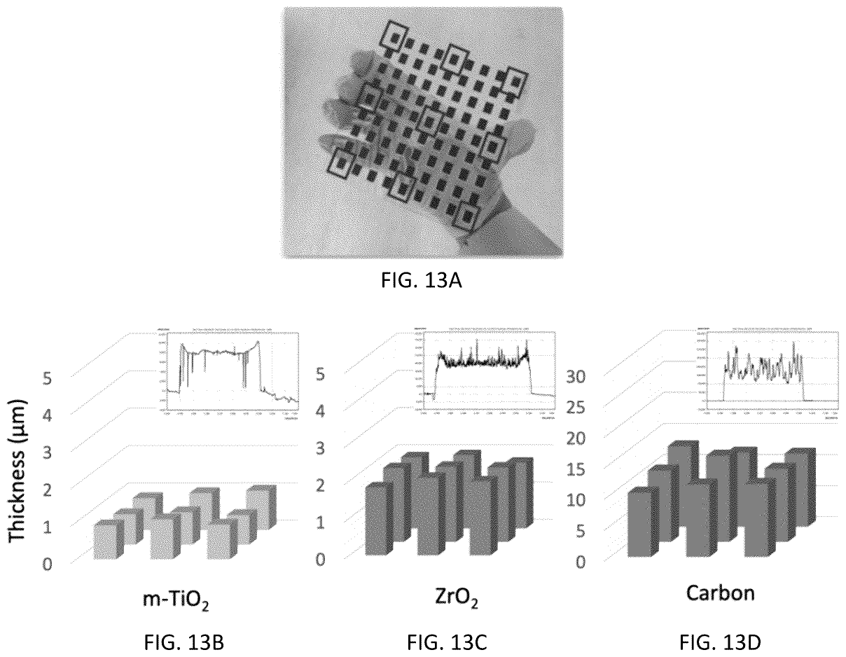

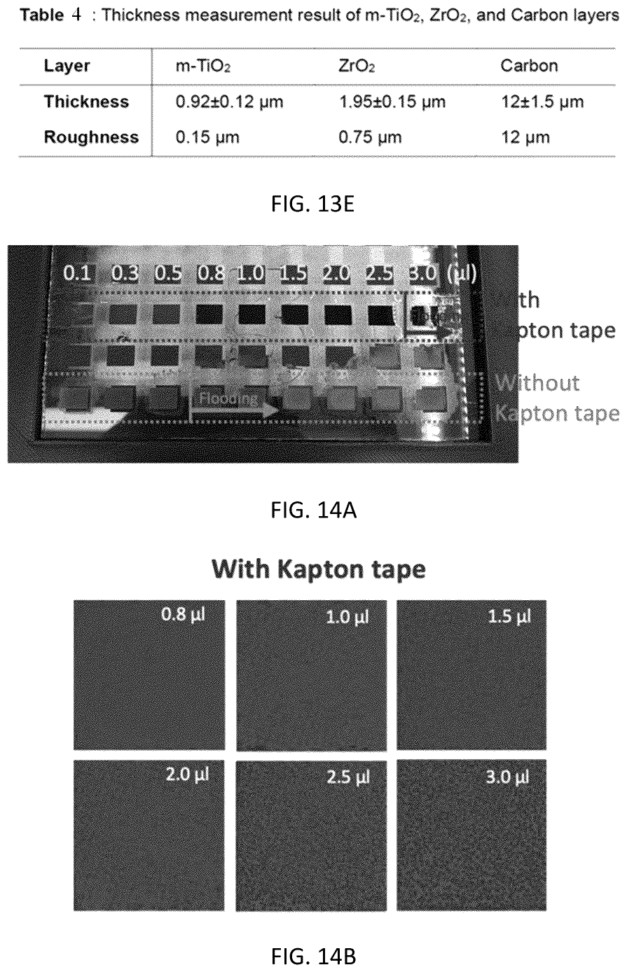

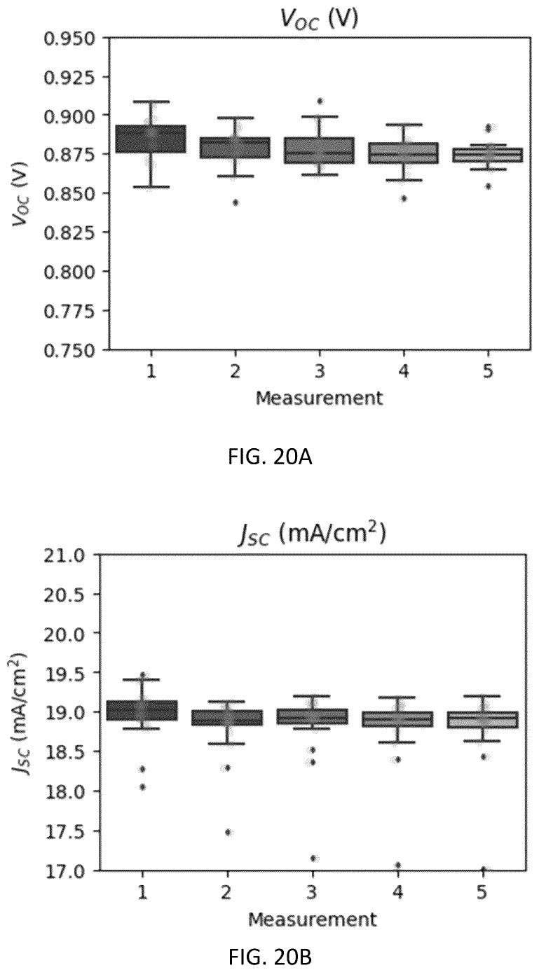

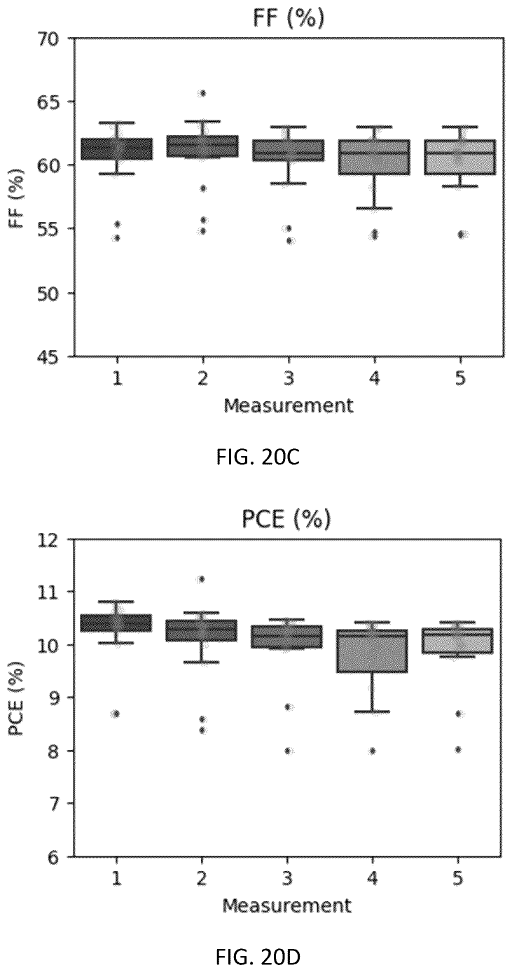

In the AI/ML-assisted experimental design module (at 360), for example, multiobjective Bayesian Optimization (BO) is used as an illustrative example to realize an optimization workflow (closed-loop optimization workflow) within the composition/additive space. After comprehensive protocol establishment (e.g., as will be discussed later below with reference to FIGS. 13A to 13D, FIGS. 14A to 14D, FIGS. 15A to 15F, FIGS. 16A to 16H, FIGS. 17A to 17E, FIGS. 18A to 18D and 19A to 19D), the HT platform according to various example embodiments achieved a PCE variance of about 0.9%, a substantial improvement compared to manual fabrication/measurement (about 4.1%) (as shown in FIG. 4B). Similar variance reduction was observed for open-circuit voltage (V_{OC}), short-circuit current density (J_{SC}), and fill factor (FF) (as will be discussed later below with reference to FIGS. 20A to 20D and Table 5 in FIG. 21), confirming the HT platform's capability for reliable data acquisition. The HT platform boosts throughput over 100-fold (10-fold faster fabrication compared to the mainstream spin coating method and 10.8-fold faster characterization than manual methods) while reducing human involvement to only 2.5% (as shown in FIGS. 4C and 4D). Furthermore, multiobjective Bayesian Optimization identified optimal additive combination regions achieving a 5.75-fold improvement in a metric that combines PCE performance and stability over pristine MAPbI₃ within two experimental iterations.

The triple mesoscopic carbon-based solar cells 312 according to various example embodiments provide an all-printed approach that yields stable devices, with the perovskite solution infiltrating the scaffold cells 312 as the last step in the device fabrication. Additives such as AVAI have been incorporated to promote interface passivation, bridge grain boundaries, and reduce ion migration. However, excessive AVAI can generate 2D perovskites due to the larger AVA⁺ cation potentially replacing MA⁺. Another oft-explored additive, MACl, is known to enhance crystallization and enlarge grain size, resulting in superior carrier transport and improved stability. Furthermore, various example embodiments found that combining both additives can exploit the advantages of robust passivation and crystallinity. In this regard, various example embodiments seek to determine whether their effects are independent or can be synergized. Prior studies investigating additives were conducted individually under conventional experimental conditions. In contrast, the high-throughput (HT), machine learning-assisted approach according to various example embodiments of the present invention is applied directly to complete devices (perovskite solar cells 313 constituting halide perovskite devices) represents a unique and unprecedented approach.

By way of an illustrative example, the application of the HT platform for exploring additives mixing space to improve the performance and stability of MAPbI₃ perovskite PV is demonstrated. 5-ammonium valeric acid iodide (HOOC(CH₂)₄NH₃I, herein referred to as AVAI) and methylammonium chloride (MACl) were chosen as target additives. The carboxylic acid group of the AVAI was found to be anchored well on the TiO₂. Minute amounts of AVAI in the MAPbI₃ enhances infiltration inside mesoporous scaffold, improving interface passivation, connecting grain boundaries, and inhibiting the ion migration. Too much AVAI is expected to form 2D perovskites since AVA⁺ is a larger cation that could replace MA⁺. On the other hand, MACl is known to assist crystallization process kinetics, resulting in an improvement in the quality of the MAPbI₃ crystals. This, in turn, leads to improvement of carrier transport and better performance stability.

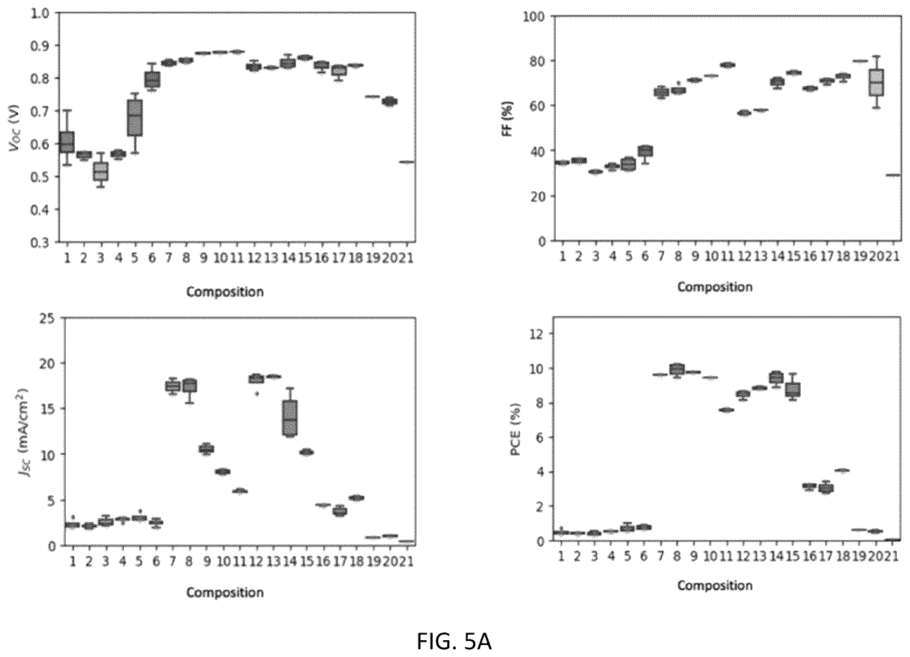

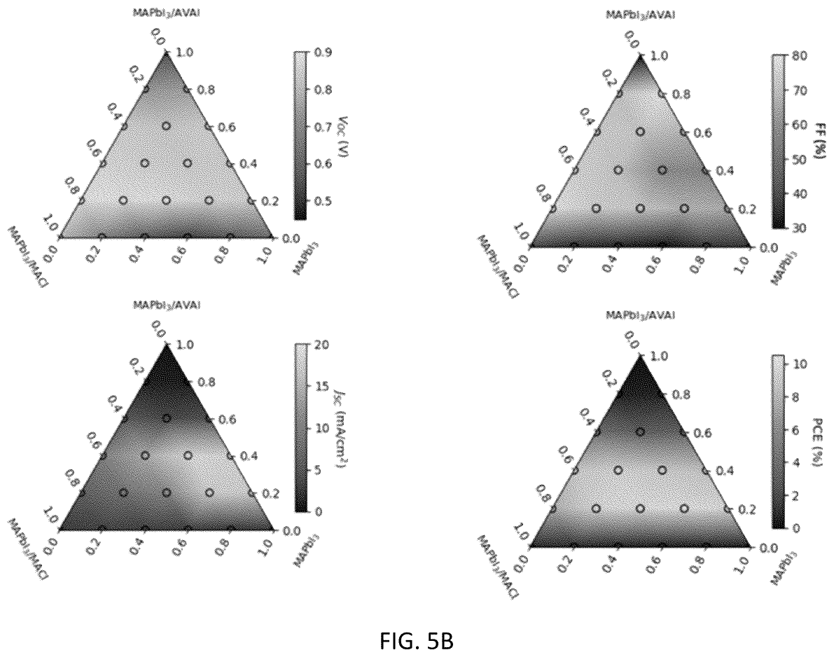

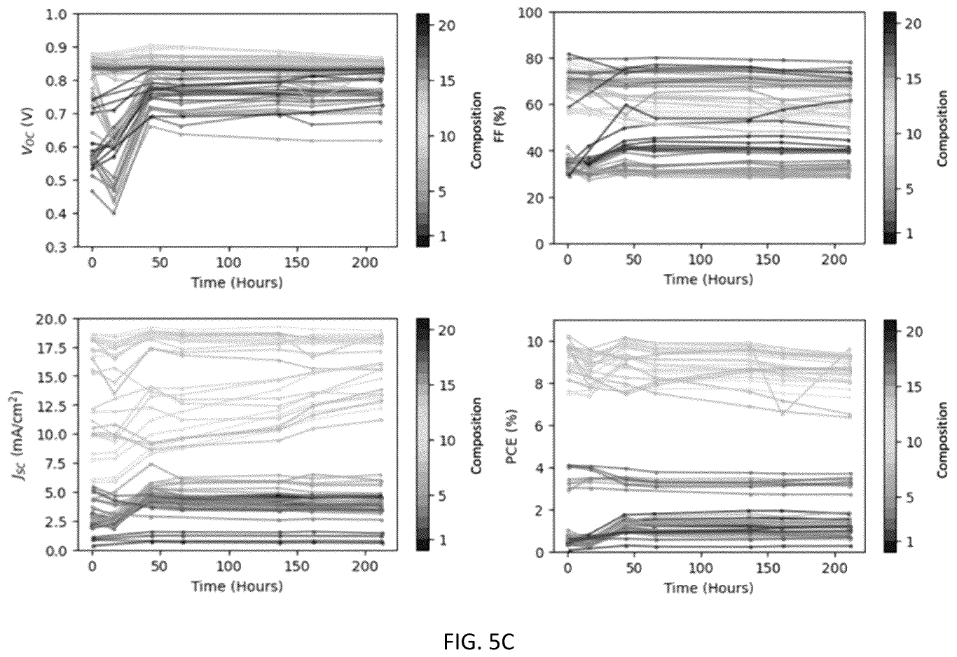

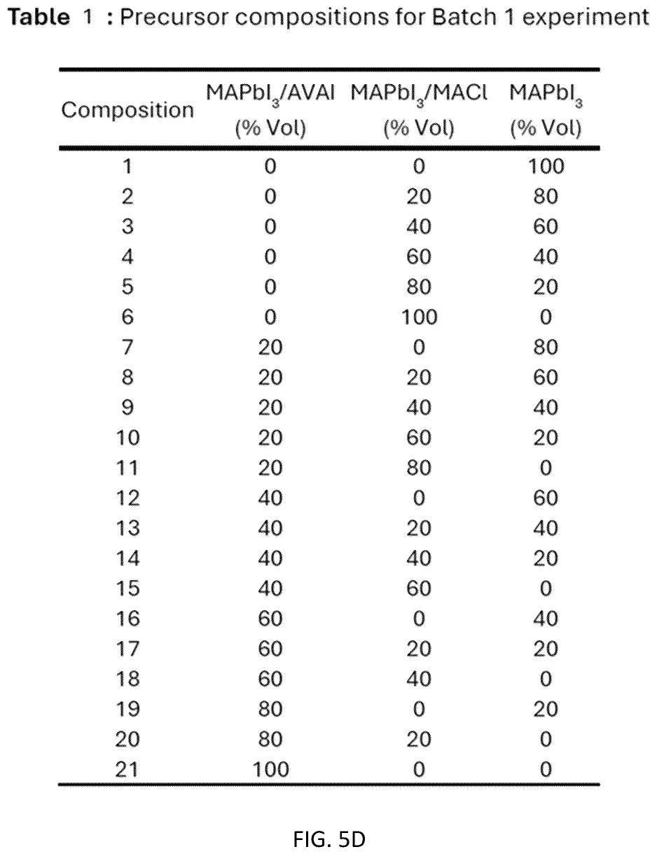

In various example embodiments, to first map out the chemical space, 21 ternary compositions derived from three mother solutions in γ-Butyrolactone (GBL)-MAPbI₃ (1.2 M)/AVAI (0.2 M), MAPbI₃ (1.2 M)/MACl (0.2 M), and pristine MAPbI3 (1.2 M) are explored using a grid sampling method (see Table 1 shown in FIG. 5D) producing 81 samples in total. In particular, Table 1 presents the precursor compositions for Batch 1 experiment, including 21 compositions with varying volume ratios of MAPbI₃/AVAI:MAPbI₃/MACl:MAPbI₃. FIG. 5A shows box plots of V_{OC}, J_{SC}, FF, and PCE extracted from HT J-V characterization. In particular, FIG. 5A shows box plots of V_{OC}, J_{SC}, FF, and PCE for different compositions (MAPbI₃/AVAI, MAPbI₃/MACl, and pristine MAPbI₃) in Batch 1, each with up to four solar cells. Data points with narrow variance and distinct profiles across compositions are observed, confirming the excellent data resolution (i.e., the narrow data spreads highlight reliable measurements). Key observations include: (i) low V_{OC} in compositions 1-6 (with no AVAI) and 19-21 (excess AVAI), (ii) high J_{SC} in compositions 7, 8, 12, and 13, which decreases as MAPbI₃/MACl increases, (iii) FF that rises with increasing MAPbI₃/MACl, and (iv) high PCE in composition with low AVAI content. Accordingly, in various example embodiments, the electrical characterization data associated with the array of perovskite solar cells 313 comprises one or more of open-circuit voltage (V_{OC}) measurement data, short-circuit current density (J_{SC}) measurement data, fill factor (FF) measurement data and power conversion efficiency (PCE) measurement data.

FIG. 5B highlights parameter gradations with the aid of a ternary heatmap. In particular, FIG. 5B shows median values in ternary space, with an interpolated heatmap showing comprehensive visualization of performance mapping across the mixing space. Each circle denotes a sampled composition, with different degree of greyscale by the median value. Greyscale interpolation between data points reveals trends such as a PCE ‘hot spot’ at 20-40% MAPbI₃/AVAI, which decreases when MAPbI₃/MACl increases. Together with FIG. 5A, these results highlight that the HT platform according to various example embodiments yields high-quality data and a comprehensive view of performance across the compositional space. FIG. 5C depicts line graphs showing device stability tests over 210 hours, which reveals unique V_{OC}, J_{SC}, FF, and PCE evolution for each composition, underscoring distinct stability behaviors.

Perovskite solar cells exhibit dynamic changes due to ionic activity, degradation, and other factors, making a single measurement insufficient. The HT platform according to various example embodiments of the present invention enables semiautomatic, time-resolved measurements to reveal how precursor compositions influence device stability. FIG. 5C depicts line graphs showing the evolution of V_{OC}, J_{SC}, FF, and PCE over 210 hours, for showing device stability tests over 210 hours, which reveals unique V_{OC}, J_{SC}, FF, and PCE evolution for each composition, underscoring distinct stability behaviors. Measurements were performed under open-air conditions, and the samples were kept in the dark between measurement intervals. Notably, compositions lacking MAPbI₃/AVAI but containing MAPbI₃/MACl exhibit an initial low V_{OC}, followed by a drop, a subsequent rise to a peak, and then a gradual decline—suggesting distinctive early stage stabilisation dynamics. Similarly, distinctive evolution features are observed in J_{SC}, FF, and PCE. This observation was made possible by the high-throughput setup, which enabled rapid device fabrication and real-time, direct comparison across devices with varied compositions.

In various example embodiments, as illustrated in FIGS. 3A and 4A, the method 300 is performed iteratively (optimizes iteratively), such as until a predefined condition is met (e.g., when the new set of different precursor compositions predicted at a current iteration is predicted or determined to be optimal (e.g., the set of different precursor compositions predicted for a next iteration is perfected to not result in device performance improvements or worsen device performance).

In various example embodiments, an optimization model is provided and configured to predict the new set of different precursor compositions for the next iteration or that the set of different precursor compositions associated with the iteration is optimal based on the electrical characterization data of each perovskite solar cell of the array of perovskite solar cells 313 associated with the iteration with respect to one or more performance objectives for said each perovskite device based on different precursor compositions comprising different perovskite source precursors of different mixing ratios associated with the iteration.

For example, multiobjective Bayesian Optimization (BO) may be used to drive the next experimental iteration. BO, an efficient method for optimizing material designs and chemical reactions, views the problem as a black-box function, using a surrogate model trained on prior results (prior evaluations) and an acquisition function to evaluate predicted gain (computes the predicted gain of a proposed solution/candidate). In particular, the optimization model is a BO model comprising one or more surrogate models (e.g., Gaussian Process (GP) surrogate models) and an acquisition function (e.g., qNEHVI acquisition function). In this regard, for each iteration of the plurality of iterations, the method 100 further comprises: training, for each of the one or more surrogate models, the surrogate model based on the electrical characterization data of each perovskite solar cell of the array of perovskite solar cells 313 associated with the iteration with respect to a corresponding performance objective of the one or more performance objectives based on different precursor compositions comprising different perovskite source precursors of different mixing ratios associated with the iteration (i.e., mixing ratios as input to each surrogate model); and predicting, using the acquisition function, the new set of different precursor compositions for the next iteration or that the set of different precursor compositions associated with the iteration is optimal based on the one or more surrogate models. Accordingly, a respective surrogate model may be trained for each performance objective. As an illustrative example for multiobjective BO (or more specifically, a bi-objective BO), MAPbI₃/AVAI, MAPbI₃/MACl, and MAPbI₃ volume ratios (mixing ratios of three different perovskite source precursors, which may simply be referred to as precursor composition mixing ratios) were selected as inputs (input parameters) thereto. Two objective outputs (output parameters) were extracted from the PCE evolution data from each perovskite solar cells 313: Peak PCE (%)—defined as the maximum PCE within 210 hours, and ΔPCE (%)—defined as the performance drop from Peak PCE to the final PCE at 210 hours (i.e., PCE₂₁₀—Peak PCE), providing a performance metric for stability. According to various example embodiments, a bi-objective optimization problem may be formulated to maximize Peak PCE and minimize ΔPCE concurrently, where the optimal solution(s) are defined at the Pareto Front. Results are quantified by the area they form with respect to a lower bound, known as the hypervolume (HV). The qNEHVI acquisition function in BoTorch was used with Gaussian Process (GP) surrogate models. Independent GPs were trained for each objective (i.e., Peak PCE and ΔPCE, respectively, using precursor composition mixing ratios as input thereto), using the reference point [0, −1.5] for hypervolume (HV) computations with respect to the normalized objective values. In this regard, each GP surrogate model may be trained on default hyperparameters with a Matern 5/2 kernel. For example, the model parameters of each surrogate model, including kernel and noise hyperparameters, are optimized by maximizing the marginal likelihood to best capture the underlying relationship and uncertainty for the respective objective. Accordingly, each GP model is trained using precursor composition mixing ratios as input descriptors and the corresponding measured objective performance (e.g., Peak PCE or ΔPCE) as outputs. Each GP model may predict the mean and uncertainty of the corresponding objective for unexplored precursor compositions (different precursor composition mixing ratios). These predictions may then be utilized by the multiobjective BO algorithm through an acquisition function (e.g., qNEHVI) to iteratively identify precursor compositions (with different precursor composition mixing ratios) that maximize the Peak PCE and minimize ΔPCE. Accordingly, in various example embodiments, the objectives may include a peak PCE objective defined to maximize a peak power conversion efficiency of perovskite solar cells 313 (with respect to precursor mixing ratios) and a PCE change (ΔPCE) objective defined to minimize a power conversion efficiency change of perovskite solar cells 313 (with respect to precursor composition mixing ratios).

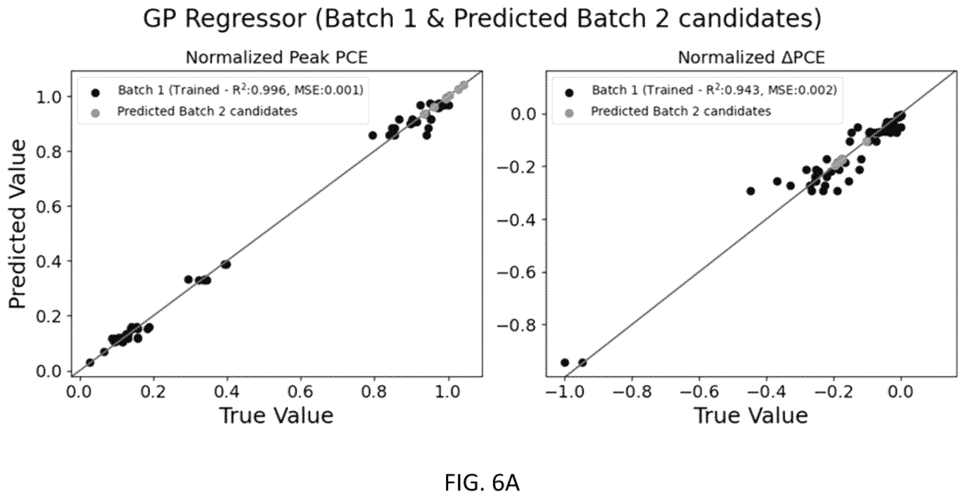

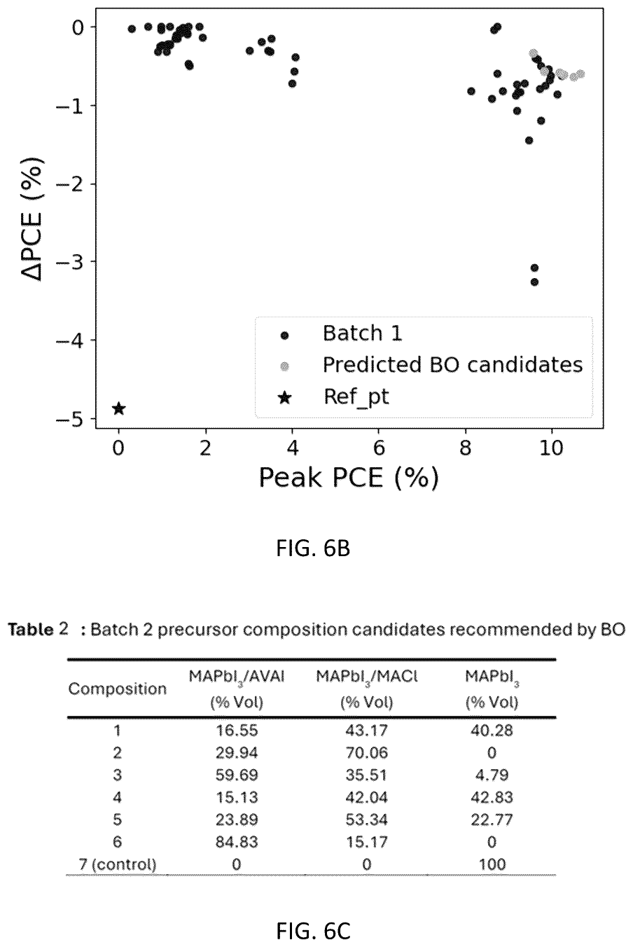

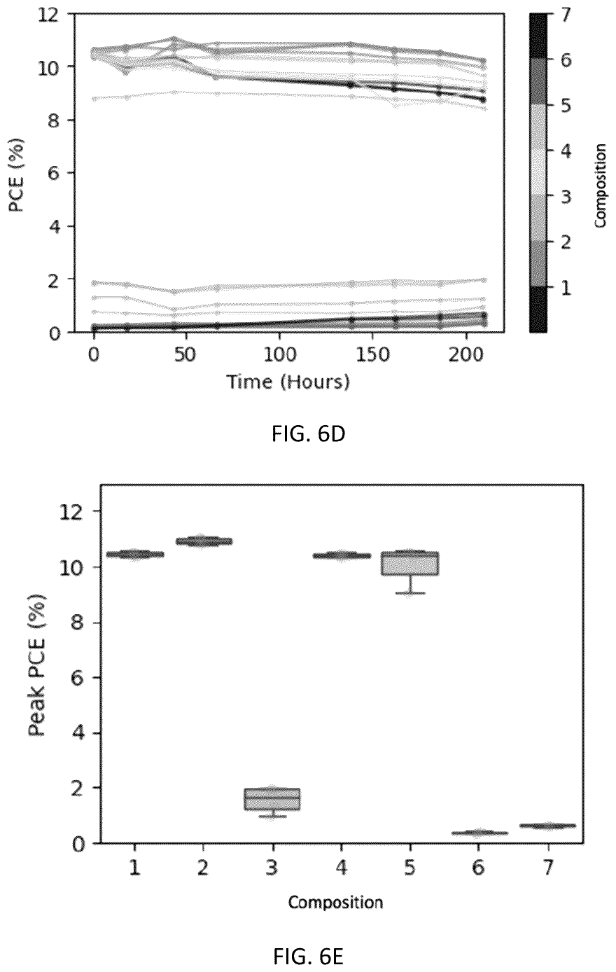

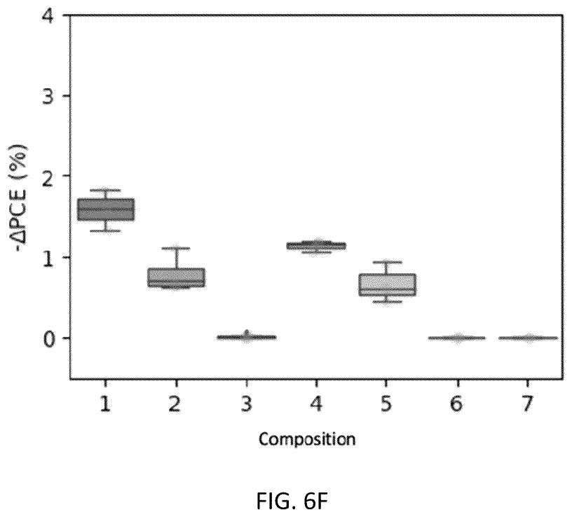

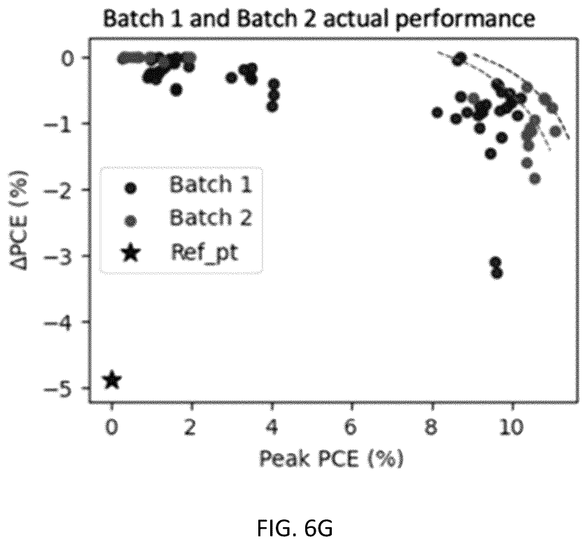

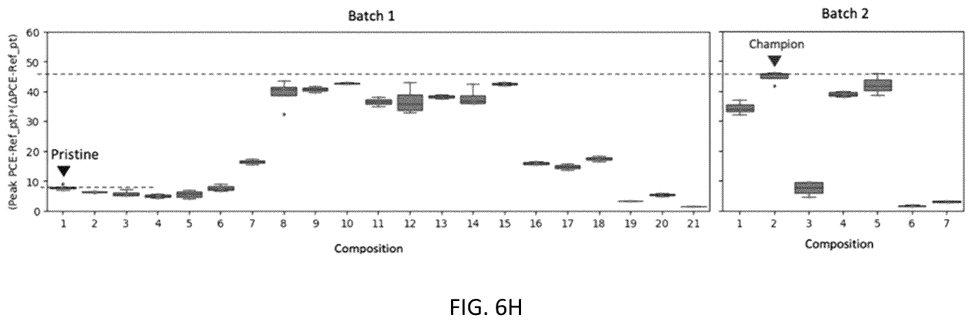

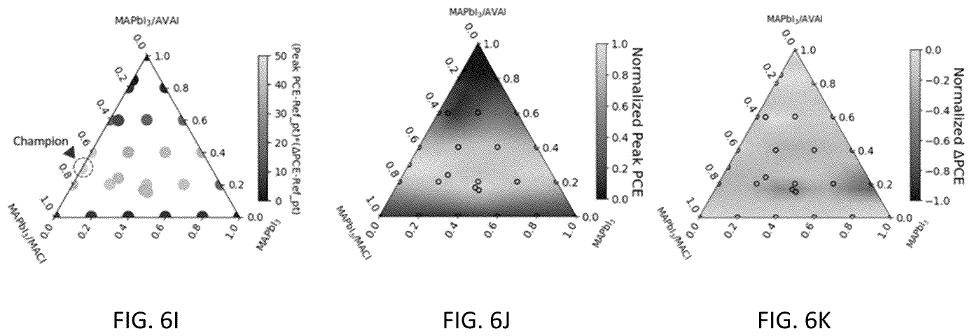

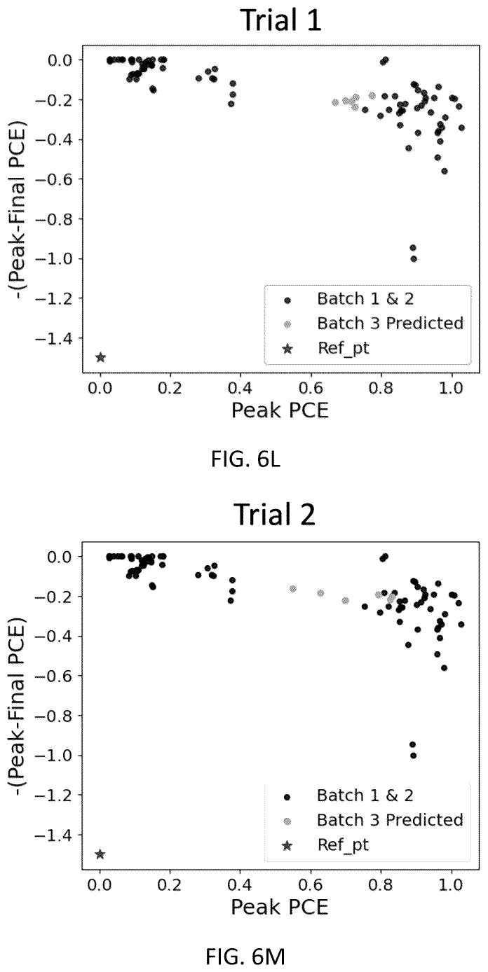

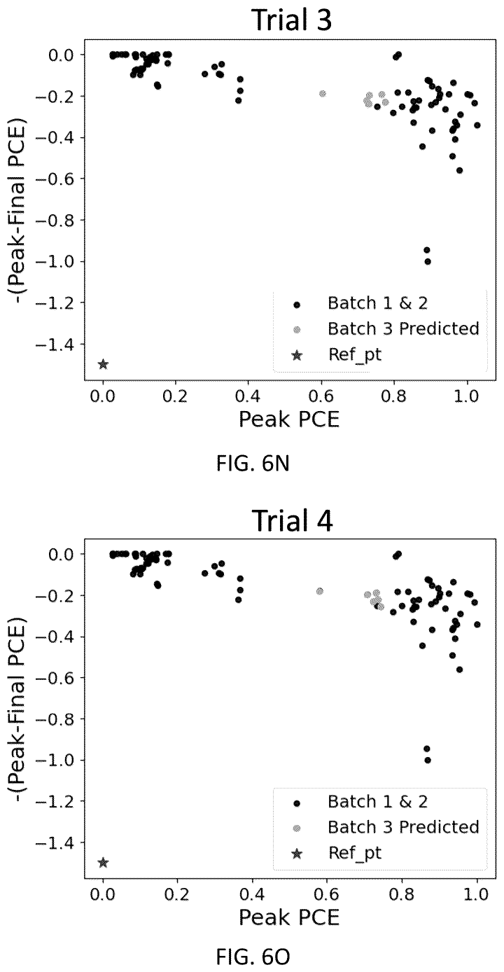

FIG. 6A depicts plots showing true (measured) vs predicted values of the GP Regressor trained with Peak PCE and ΔPCE from Batch 1 (darker shaded dots) and predictions for six new candidates (lighter shaded dots). In particular, FIG. 6A compares the measured (darker shaded dots) vs predicted values (lighter shaded dots) from the GP Regressor, with normalized Peak PCE on the left and ΔPCE on the right. High R² (0.996 for Peak PCE and 0.943 for ΔPCE) and low mean-squared error (MSE) (0.001 and 0.002, respectively) indicate high model accuracy. The BO workflow then proposed six new candidates for the next iteration or batch (Batch 2) (e.g., corresponding to the new set (or next set) of different precursor compositions (with different precursor composition mixing ratios) for the next iteration (e.g., Batch 2) described hereinbefore), shown as the lighter shaded dots. FIG. 6B reveals these candidates may achieve a Peak PCE of around 11% while keeping ΔPCE moderate. In particular, FIG. 6B shows the peak PCE and ΔPCE for actual measurements of Batch 1 and predicted Peak PCE and ΔPCE for BO candidates. Batch 2 solar cells, with new precursor compositions listed in Table 2 shown in FIG. 6C, were fabricated and characterized using the HT platform. In particular, Table 2 presents the Batch 2 precursor composition candidates predicted or recommended by BO. Four cells were produced per composition to ensure reliable data (improve the statistical confidence in the data), and 100% pristine MAPbI₃ was included as a control (i.e., a control sample). In this regard, the computing system 350 may set the automated liquid handling robot 201 with precursor composition set information on the set of different precursor compositions (with different precursor composition mixing ratios) associated with the iteration (e.g., corresponding to Batch 2) (e.g., by sending/communicating the precursor composition set information or data to the automated liquid handling robot 201 based on wired or wireless communication) for the automated liquid handling robot 201 to produce the perovskite solutions according to the set of different precursor compositions (with different precursor composition mixing ratios) associated with the iteration and infiltrate each solar cell of the array of solar cells 312 formed on the substrate 310 associated with the iteration with the perovskite solution having the selected precursor composition from the set of different precursor compositions associated with the iteration to form a corresponding perovskite device, resulting in the array of perovskite solar cells 313 associated with the iteration. The computing system 350 may then control the automated electrical measurement probing robot 202 to perform electrical characterization on each perovskite solar cell of the array of perovskite solar cell 313 (e.g., one by one) for generating electrical characterization data associated with the array of perovskite solar cells associated with the iteration.

Peak-PCE and ΔPCE were extracted from the PCE evolution data in Batch 2 (see FIGS. 6D to 6F). In particular, FIGS. 6D to 6F show the performance of Batch 2 whereby FIG. 6D shows the PCE evolution over 210 hours; FIG. 6E shows the Peak PCE and FIG. 6F shows the −ΔPCE per Composition. It shows that Batch 2 Composition 2 achieved the highest Peak PCE while the −ΔPCE is in the midrange. FIG. 6G compares Peak-PCE vs ΔPCE for Batches 1 and 2, showing that Batch 2 successfully pushed the Pareto front to higher performance. In particular, FIG. 6G shows experimental results plotted in Peak PCE vs ΔPCE space which shows Batch 2 notably expanding the Peak PCE×ΔPCE hypervolume relative to Ref_pt. Composition 2 in Batch 2 reached about 11% PCE (see FIG. 6E) with a ΔPCE of about −0.7% (see FIG. 6F).

FIG. 6H shows cumulative hypervolume (HV) (Peak PCE×ΔPCE relative to Ref_pt) for cells in both batches highlights Composition 2 in Batch 2 as the champion, outperforming pristine MAPbI₃ by over five times. In this regard, the HV calculations (FIG. 6H) confirm that Batch 2 Composition 2 outperforms all others, yielding a cumulative achievement 5.75 times higher than pristine MAPbI₃.

FIG. 6I shows median values from FIG. 6H plotted in ternary space, with the champion composition identified. In particular, FIG. 6I illustrates the HV gradation across the ternary space, indicating that 20-40% MAPbI3/AVAI yields strong performance. Batch 2 data was fed back into the model for subsequent recommendations. Despite high accuracy, the BO-suggested compositions were predicted to underperform compared to earlier achievements (see FIGS. 6L to 6O). This indicates the compositional space was thoroughly explored. In particular, FIGS. 6L to 6O show BO recommendation for Batch 3 and their predicted achievements (Peak PCE and −(Peak−Final PCE), in several trials. Consequently, it was concluded that the optimum was reached and opted not to proceed with another batch (e.g., corresponding to the AI model (or optimization model) predicting or determining that the set of different precursor compositions associated with the iteration (e.g., corresponding to Batch 2) is optimal described hereinbefore according to various embodiments of the present invention).

FIGS. 6J and 6K show the final models for normalized Peak PCE and ΔPCE, respectively. Black circles mark Batch 1 and Batch 2 data points, while the greyscale map represents the model's view of the compositional space. In particular, FIGS. 6J and 6K show the final models for Peak PCE and ΔPCE in normalized values reveal AVAI significantly enhances Peak PCE at 0.2-0.4 volume ratio (0.04-0.08 M AVAI), while MACl strongly affects ΔPCE in regions with favorable Peak PCE. In FIG. 6J, AVAI strongly influences Peak PCE within a 20-40% volume ratio (0.04-0.08 M), especially when MAPbI₃/MACl is high (70%), yielding optimal additive concentrations at 0.14 M MACl and 0.06 M AVAI. Notably, the optimal MACl molar ratio is lower than those reported in previous studies, likely due to synergistic effects from the co-addition of AVAI. FIG. 6K indicates MACl primarily affects ΔPCE within this same 20-40% AVAI range, where it helps keep ΔPCE near zero. These findings reveal an interdependency between AVAI and MACl and provide valuable insights on tailoring favorable combinations of MAPbI₃/AVAI and MAPbI₃/MACl concentrations that optimize Peak PCE and maintain long-term stability for MAPbI₃ perovskite solar cells.

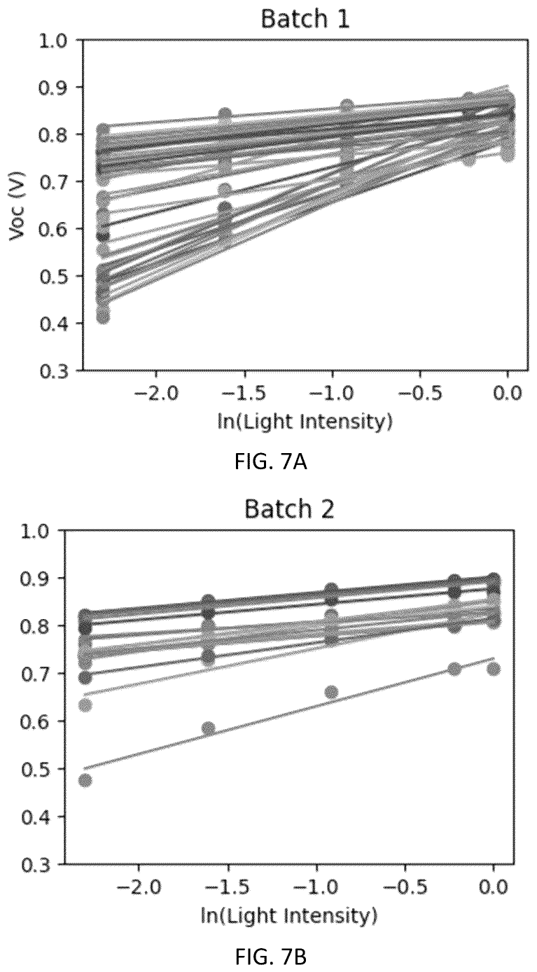

The high-throughput platform according to various example embodiments also allows for additional characterization using light intensity-dependent V_{OC} measurements to identify dominant charge recombination pathways in all of the compositions. Trap-assisted (Shockley-Read-Hall, SRH) recombination often limits performance, and the ideality factor (n_{ID}), derived from V_{OC}-light intensity data, indicates whether recombination is predominantly SRH (n_{ID}≈2) or bimolecular radiative (n_{ID}≈1). FIGS. 7A and 7B show V_{OC} versus ln(Light intensity) plots (Light intensity dependence V_{OC} measurement result) for Batches 1 and 2, respectively, from which n_{ID} was extracted.

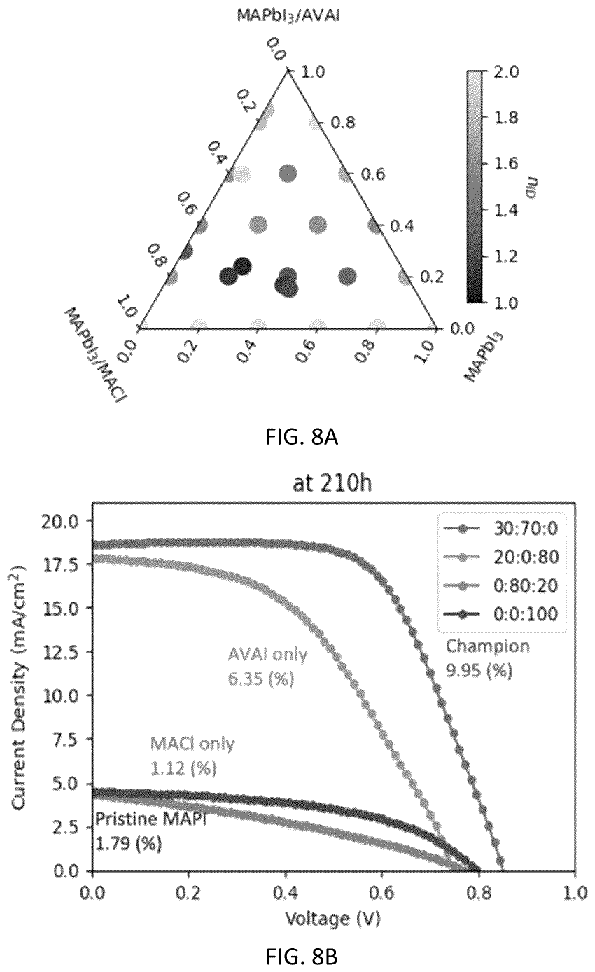

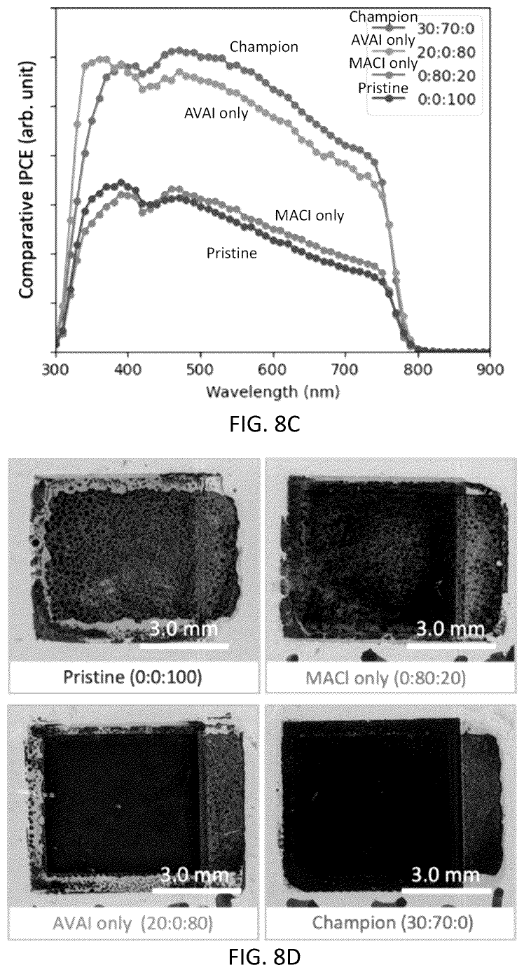

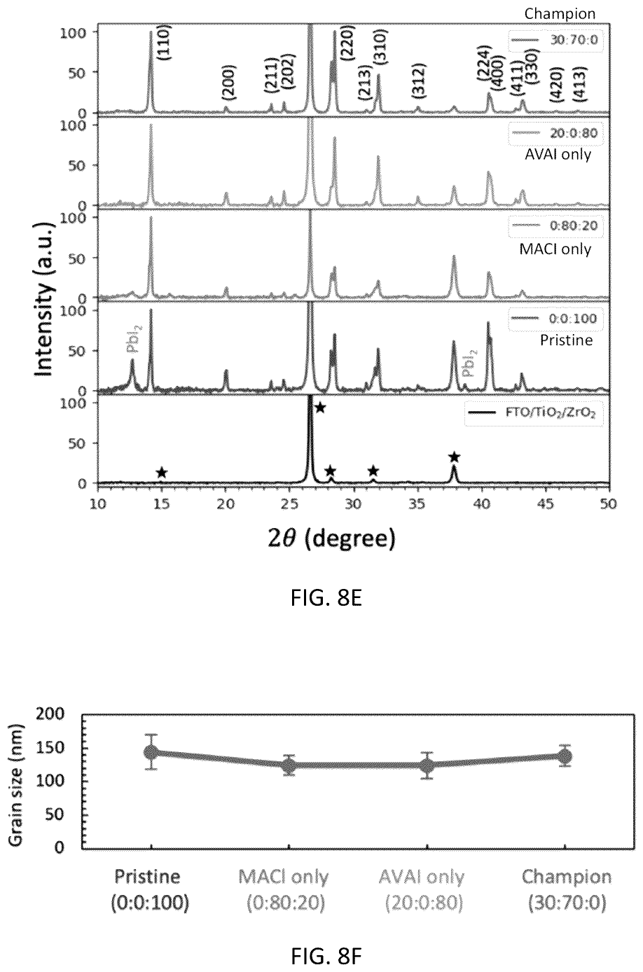

FIG. 8A illustrates a ternary plot of median n_{ID} values, revealing lower n_{ID} in regions with low MAPbI₃/AVAI ratios. Within these zones, higher MAPbI₃/MACl ratios drive n_{ID} closer to 1, indicating fewer intermediate states and more direct band-to-band recombination. The n_{ID} distribution aligns with the Peak PCE and ΔPCE findings, corroborating the effectiveness of low AVAI and high MACl concentrations in enhancing device performance. In particular, in FIG. 8A, ideality factor n_{ID} extracted from light intensity dependence V_{OC} measurement of Batch 1 and Batch 2 is plotted in ternary space. n_{ID} close to 1 in the optimal AVAI range and higher MACl volume ratios highlight dominant band-to-band recombination and fewer intermediate states.

Next, four representative samples—30:70:0 (Champion), 20:0:80 (AVAI-only), 0:80:20 (MACl-only), and 0:0:100 (Pristine)—were selected for high fidelity experiments. FIG. 8B shows their J-V curves at 210 hours. In particular, FIG. 8B shows J-V characteristics of selected samples, including a champion device (AVAI+MACl), AVAI-only, MACl-only, and pristine devices at 210 hours. The legend shown in FIG. 8B denotes the MAPbI₃/AVAI:MAPbI₃/MACl:MAPbI₃ volume ratio. FIG. 8C shows the comparative Incident Photon-to-Current Efficiency (IPCE), which shows AVAI enhances short-wavelength IPCE, while MACl improves long-wavelength IPCE. The comparative IPCE data reveal that samples with AVAI (Champion and AVAI-only) outperform the others across all wavelengths, especially from 300-450 nm. FIG. 8D shows digital photographs of cells after nine months, where visible PbI₂ degradation product (yellow areas) is minimal in the champion device. Photographs from the glass side (FIG. 8D) show uniform dark areas in AVAI-containing cells, indicating improved perovskite infiltration to the TiO₂ layer and enhanced carrier extraction, as supported by the IPCE results. FIG. 8E shows 2θ-XRD patterns of fresh selected samples and the scaffold. In particular, FIG. 8E shows the 2θ-XRD patterns of four representative samples (measured from the ZrO₂ side after carbon removal, postfabrication). AVAI-containing samples exhibit lower PbI₂ peak intensities compared to the Pristine and MACl-only samples. FIG. 8F shows grain size of perovskite crystals in the selected samples calculated from the XRD peak after Rietveld refinement. While prior studies indicate that MACl primarily improves crystallization and grain growth, various example embodiments found that the addition of 5-AVAI provides a greater impact on the crystallization process. This effect is likely attributed to the bulky 5-AVA⁺ cation, which is unlikely to incorporate into the 3D perovskite lattice and instead preferentially segregates to grain boundaries and surfaces during crystallization. Its accumulation at the crystal periphery likely moderates the crystallization kinetics, effectively encapsulating the growing grains. Consequently, this promotes more complete phase conversion, as evidenced by reduced residual PbI₂ during the early stages of growth. In contrast, MACl appears to enhance long-term stability of the perovskite, as indicated by the stability measurements: FIG. 6K shows improved performance retention, while FIG. 8D reveals suppressed visible yellowish PbI₂ formation in the MACl containing samples after nine months. The black central area remains intact in all samples, likely due to the carbon layer's hydrophobic protection from the carbon layer.

Previous theoretical work suggests that Cl⁻ propagation to grain boundaries can effectively passivate shallow defect levels near the valence band maximum. These passivated grain boundaries can impede degradation pathways involving H₂O and O₂ reactions. Consequently, as reflected in the experimental results, samples with MACl exhibit lower n_{ID} values and improved stability. This passivation also benefits IPCE from 450-800 nm; as shown in FIG. 8C, cells with MACl (Champion vs AVAI-only, MACl-only vs Pristine) slightly outperform those without. Although MACl can enlarge MAPbI3 grains, the XRD data (calculated with Bruker TOPAS) show similar grain sizes (about 120-150 nm) in all samples (FIG. 8F), likely due to scaffold pore constraints. However, MACl-containing cells (MACl-only, Champion) exhibit a higher (110) crystal orientation, potentially enhancing carrier generation and transport.

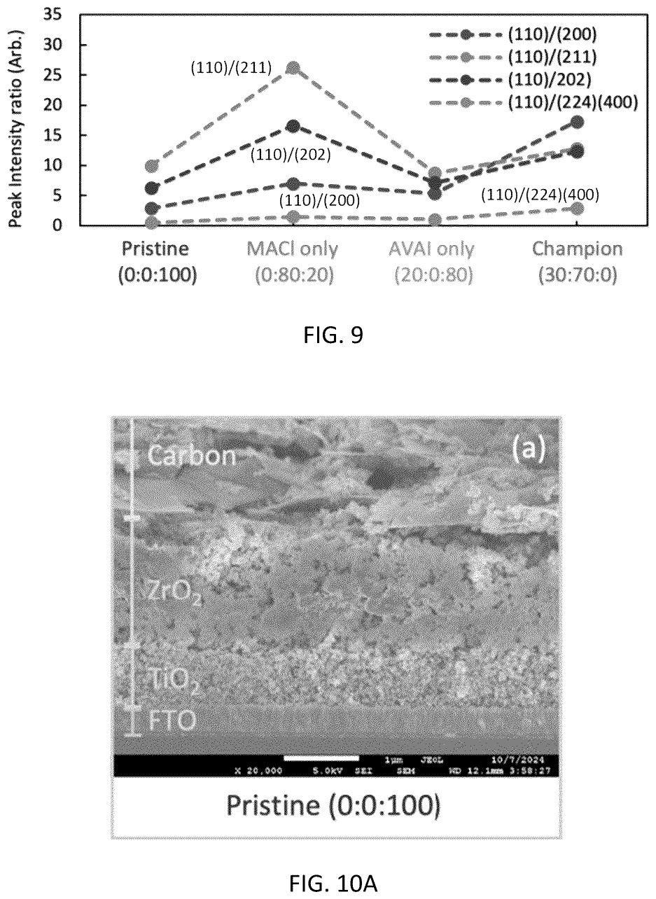

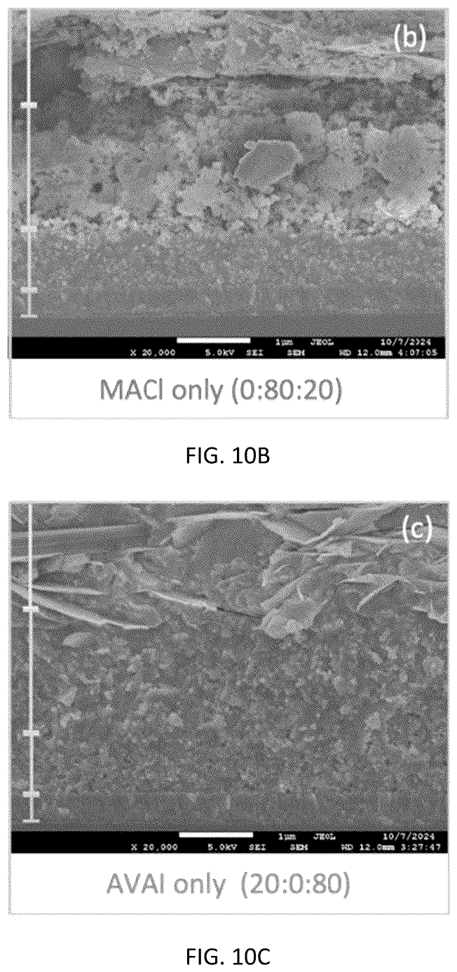

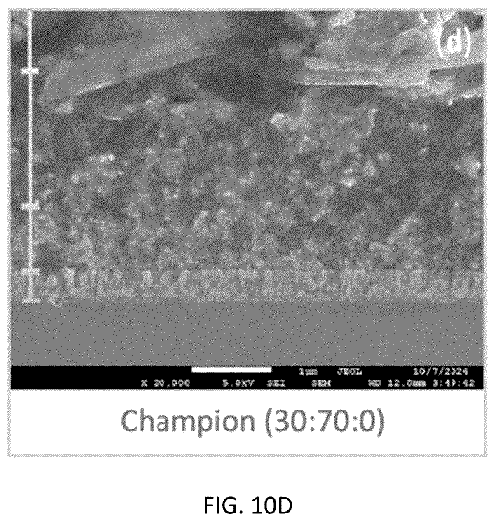

FIG. 9 shows the intensity ratio of (110) peak as the dominant peak compares to other non-dominant peaks of selected samples. FIGS. 10A to 10D show the cross-sectional SEM image of (a) pristine sample, (b) MACl only, (c) AVAI only, and (d) Champion sample, respectively.

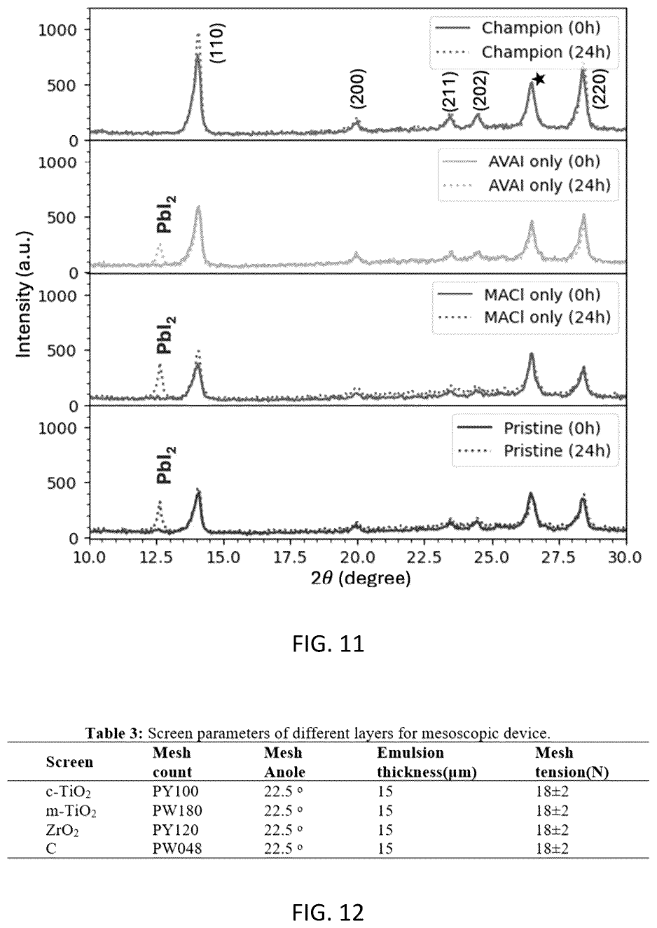

The transferability of the recipes to spin-coated films was preliminarily assessed, revealing similar stability trends, as shown in FIG. 11. In particular, FIG. 11 shows the 2θ-XRD patterns of planar samples after removal from glovebox (0 h) and after 24 hours of exposure in ambient air (24 h). Developing an HTE workflow specifically tailored for planar architectures would be valuable for enabling efficient and systematic exploration in spin-coated device configurations. In summary, various example embodiments developed a HT platform that allows rapid screening of precursor compositions directly in device configurations. The HT approach according to various example embodiments of the present invention accelerates experimentation by over 100-fold, significantly reduces human involvement, and reduces data variance to 25% compared to manual work. Various example embodiments demonstrated the capability of the HT platform and combined it with multiobjective BO to identify optimum additive compositions for balanced PCE and stability, achieving a 5.75-fold improvement metric within only two batches of experiments.

As an illustrative example, the high-throughput exploration of MACl and 5-AVAI as mixed additives in MAPbI₃ also reveals new mechanistic insights. While prior studies indicate that MACl primarily improves crystallization and grain growth, various example embodiments found that 5-AVAI provides a greater impact on the early crystallization process. MACl, on the other hand, appears to hinder degradation over the long-term. Taken together, these findings demonstrate that the combined use of 5-AVAI and MACl yields perovskite films with improved initial crystalline quality (minimal PbI₂-related secondary phase) and enhanced long-term stability (resistance to PbI₂ formation over time), which was not achievable by either additive alone.

The results also demonstrated that high-resolution data and a more holistic understanding of the performance gradation across mixing space can be obtained. This opens numerous potential applications for various experimentation, especially related to precursor formulations, such as the exploration of compositional space of halide perovskite itself, a wider range of additives screening, and the pursuit of toxic-free precursors. Accordingly, the method 300 of optimizing material compositions for halide perovskite devices according to various example embodiments of the present invention further accelerates the development of perovskite PV technology.

For better understanding, various aspects of the method 300 (or the corresponding HT platform) for optimizing material compositions will now be described in further details as illustrative examples according to various example embodiments of the present invention.

### Device Fabrication and Characterization

### Materials

Blocking Layer TiO₂ paste (BL-1), methylammonium iodide (MAI), and 5-ammonium valeric acid iodide (5-AVAI) were purchased from Greatcell Solar. Terpineol from Sigma-Aldrich was used to dilute TiO₂ paste in the weight ratio of 1:1.4. TiO₂ paste (NRD30) was purchased from Greatcell Solar. ZrO₂ paste (Zr-Nanoxide ZT/SP) was purchased from Solaronix. Carbon paste was purchased from Hubei Wonder Solar LLC. Methylammonium chloride (MACl) was purchased from Merck Millipore Corporation. Lead iodide (PbI₂) was purchased from the Tokyo Chemical Industry. γ-butyrolactone (GBL) as a solvent for perovskite precursors was purchased from Sigma-Aldrich.

### Scaffold Screen Printing Preparation

10×10 cm² pre-patterned FTO substrates were initially ultrasonicated in a decon soap solution, deionized water, and then ethanol, with each cleaning cycle lasting 30 minutes. Post-cleaning, the substrates were subjected to drying using an argon gas blower. A compact layer of TiO₂ was then deposited via screen printing on the prepared substrates using a MicroTec MT320TV screen printer, followed by an annealing process at 500° C. for 30 minutes, with the temperature increasing at a rate of 40 minutes. Afterward, a layer of mesoporous TiO₂ (about 1.00 μm thick) and subsequently, an about 2 m layer of mesoporous ZrO₂ was screen printed. Each of them was annealed at 500° C. for 30 minutes with 40 minutes ramping rate. Lastly, a mesoporous carbon electrode was screen printed and annealed at 400° C. for 30 minutes, with a ramping rate of 30 minutes. 10×10 cm² substrates with 81 scaffold cells on each were then ready to be infiltrated with perovskite precursors. All the printing processes were conducted in an open-air environment (45±20% RH, 25±2° C.). For example, Table 3 shown in FIG. 12 shows example screen parameters of different layers for the mesoscopic device according to various example embodiments of the present invention.

Prior to printing, the conducting part of the FTO glasses undergoes laser etching to create resistive parallel horizontal and vertical lines (with a width of 200 μm), effectively separating the p-side and n-side, and separating each device. Each layer's screen or mesh is custom-designed with parameters outlined in Table 3 so that each layer reaches the targeted thickness and is printed in the desired shape and position. The printing parameters are consistently maintained as follows: Printing pressure ranging from 0.173 to 0.175 Mpa, printing gap set between 0.2 to 0.3 mm, and printing speed at 100 mm/s.

An example detailed process of the scaffold preparation is as follows.

Substrate cleaning: Laser-etched FTO substrates are cleaned with decon soap solution, DI water, and Ethanol by 30 min of sonication for each. After the last sonication, the substrates are rinsed with ethanol followed by nitrogen drying.

c-TiO₂ layer printing: Screen print the compact TiO₂ (c-TiO₂) into the substrate. After printing, the wet film is relaxed at Room Temperature (RT) for the removal of the bubbles and to achieve a uniform thin film. The substrate then sintered to 500° C. for 30 minutes at a ramp rate of 40 minutes.

m-TiO₂ layer printing: The TiO₂ paste is diluted with terpineol with a weight ratio of 1:1.4. The diluted paste is sonicated and stirred before use. Following the printing process, the wet film undergoes a 15-minute relaxation period at room temperature to achieve a consistently even surface, followed by a 15-minute drying phase at 80° C. The substrate was then sintered at 500° C. for 30 minutes at a ramp rate of 40 minutes.

ZrO₂ layer printing: The ZrO₂ paste is printed and then relaxed for 15 minutes at RT, followed by drying at 80° C. for 15 minutes. It then undergoes sintering at 500° C. for 30 minutes at a ramp rate of 40 minutes.

Carbon layer printing: A Carbon paste is printed and then relaxed for 15 minutes at RT, followed by drying at 80° C. for 15 minutes. It then undergoes sintering at 400° C. for 30 minutes with a ramp rate of 40 minutes.

These procedures, employing a custom-designed screen/mesh, are capable of producing 81 scaffold cells on a 10×10 cm² FTO glass substrate, with each cell having an active area of 5×5 mm².

Subsequently, perovskite source precursors are prepared manually. A liquid handling robot, Opentrons OT2, is employed for the precise mixing of perovskite source precursors and subsequent drop-casting into the scaffold substrates. Opentrons OT2 features a Python API, enabling scientists to design custom experiment protocols using Python programming language.

The infiltrated substrate undergoes annealing to crystalize the perovskite. For MAPbI₃ system in GBL as a solvent, the device undergoes 50° C. for 90 minutes, followed by 60° C. for 30 minutes.

### Perovskite Precursors Preparation and Infiltration

For HT platform testing, MAPbI₃ with AVAI as an additive was prepared by dissolving 1.2 M of PbI₂, 1.2 M of MAI, and 0.045 M of AVAI in GBL.

For HT experimentation, three ‘mother’ solutions were prepared: 1) 1.2 M of PbI₂, 1.2 M of MAI, and 0.2 M of AVAI in GBL, 2) 1.2 M of PbI₂, 1.2 M of MAI, and 0.2 M of MACl in GBL, and 3) 1.2 M of PbI₂ and 1.2 M of MAI in GBL. The solutions were stirred on a hot plate heated at 50° C. for at least one hour and then filtered through a 0.45 μm PTFE filter. These three mother solutions collectively define a ternary mixing space, encompassing compositions ranging from 100% MAPbI₃(1.2 M)/AVAI(0.2 M), 100% MAPbI₃(1.2 M)/MACl(0.2 M), to 100% pristine MAPbI₃(1.2 M) at the extremities.

Liquid handling robot Opentrons OT2 was programmed to distribute the three mother solutions into 2 ml polypropylene vials sitting on a 50° C. temperature module, to make solutions with different mixing ratios according to the precursor composition set information on the set of different precursor compositions (with different precursor composition mixing ratios) for the corresponding iteration. A solution mixing protocol was done in each vial before subsequently drop-casting 2 μl of the solution onto a 0.25 cm² scaffold cell. After infiltrating scaffold cells (e.g., 81 scaffold cells), the substrate then underwent annealing at 50° C. for 90 minutes, followed by subsequent annealing at 60° C. for an additional 30 minutes to crystallize the perovskite. Precursor mixing, infiltration, and annealing were conducted in the open-air environment (45±20% RH, 25±2° C.).

### Characterization

The J-V characterization was conducted on an in-house probing robot employing a repurposed Ender-3 V2 3D printer, Keithley 2450 as a Source Measurement Unit, and G2V Pico (class AAA) as a Solar simulator. Shadow masks of various sizes made from a thin metal plate featuring 81 square holes were designed to align (e.g., precisely align) with the positions of 81 cells on a substrate. IPCE measurements were carried out using the Newport model SPCS260-USB-QEPVSI. The X-ray diffraction (XRD) patterns were collected on a Bruker D8 Advance diffractometer with Cu Kα radiation. The carbon layer was peeled off using Kapton tape right before the XRD measurement, exposing the surface of the ZrO₂ porous layer with perovskite crystal in it.

### Platform Testing and Experimental Protocol Establishment

### Scaffold Thickness Homogeneity

Scaffold thickness homogeneity is an important physical parameter to ensure that the device characteristics across the substrate are not heavily affected by the scaffold's layer thickness differences. The thickness of the layer was measured by a profilometer at nine positions as seen in FIG. 13A to examine the thickness homogeneity. There is no noticeable position dependency of the thickness. The average thickness and the roughness were then calculated and extracted in Table 4 shown in FIG. 13E. In particular, Table 4 in FIG. 13E present the thickness measurement results of m-TiO₂, ZrO₂, and Carbon layer. The ±error of the thickness are all lower than the roughness per measurement. Consequently, the thickness of all the layers can be considered reasonably homogeneous across the substrate surface. It is noteworthy that the carbon layer exhibited a consistently rough morphology. In particular, FIGS. 13A to 13D illustrate scaffold thickness homogeneity, whereby in FIG. 13A, the boxed squares show the cell positions that undergo thickness measurements, and FIGS. 13B to 13D show thickness measurement results of m-TiO₂, ZrO₂, and Carbon layer, respectively. The inset shows the representative surface profile of one cell.

### Precursors Infiltration Volume Optimization

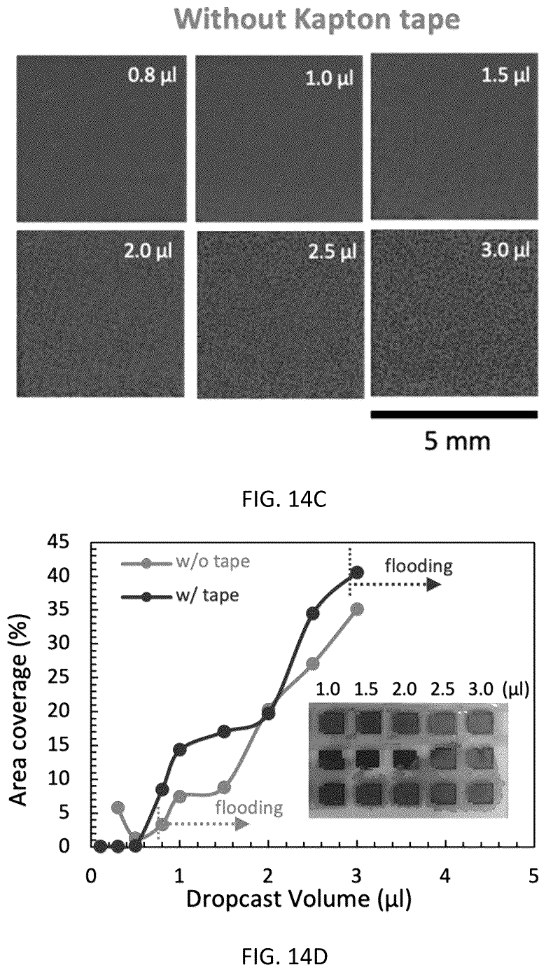

FIGS. 14A to 14D show precursors volume optimization for infiltration. In particular, FIG. 14A shows scaffold cell infiltration with and without Kapton-tape as a confinement, with different volumes. FIGS. 14B and 14C show the area coverage of perovskite from the back side (glass) of cells with Kapton tape and without Kapton tape respectively. The red area is the image-processed area indicating the presence of perovskite on the bottom layer. FIG. 14D show the plot of Area coverage (%) against drop cast volume. The inset shows the image from the top (carbon) side shows visible precipitation on the carbon side with volume of more than 2 μl. A perovskite solution of 2 μl with Kapton-tape confinement was selected as a standard protocol for infiltration owing to its minimum precipitations on the carbon surface while maintaining the highest area coverage before flooding the neighboring cells.

### Scaffold Resistance Homogeneity

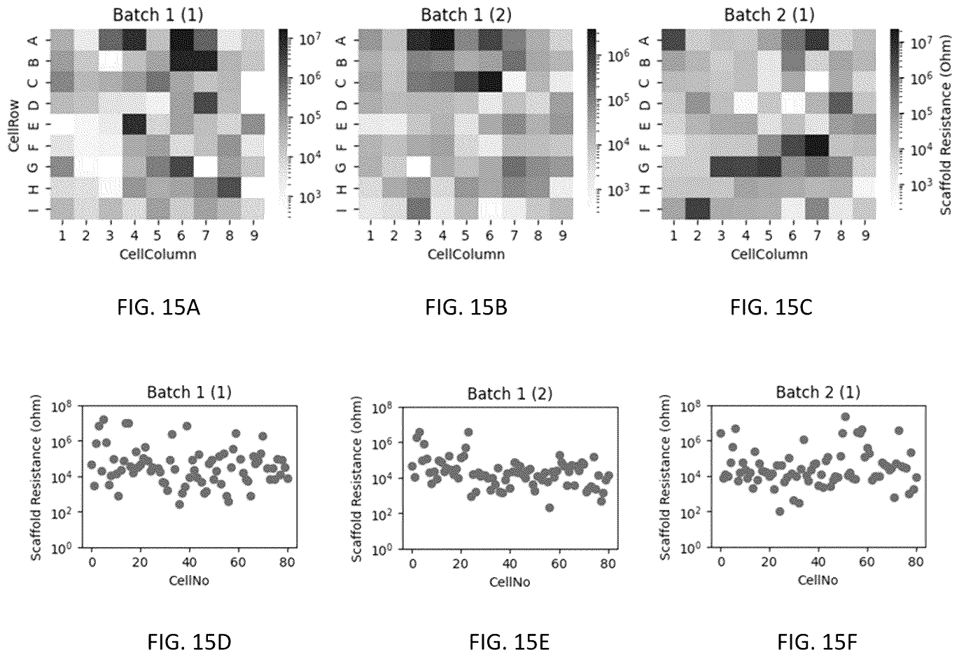

This analysis aims to assess the electrical characteristics homogeneity right before perovskite infiltration. FIGS. 15A to 15F illustrate scaffold resistance homogeneity. In particular, FIGS. 15A to 15C show scaffold resistance of Batch 1 (1), Batch 1 (2), and Batch 2 (1) according to the cell's position on the substrates. FIGS. 15D to 15F show the scaffold resistance according to cell numbers counted from cell A1 to I9. The results in FIGS. 15A to 15C show that there is no positional dependency on the scaffold resistance. FIGS. 15A and 15B are scaffolds of the same batch and FIG. 15C is from a different batch. Across all 81 cells of these three substrates, the resistance values were randomly distributed without any noticeable positional trends. Ideally, the layers should be insulators (R>10⁸ Ohm). However, as seen in FIGS. 15D to 15F, the value of the scaffold resistance is ranged between 10² to 10⁸ (Ohm). This variation may be attributed to the inherent challenges of controlling the interface border between each layer in screen-printing deposition. For instance, a minute amount of carbon may infiltrate through porous ZrO₂, potentially resulting in a short circuit between the Carbon and TiO₂ layers. While the randomness and variation in Scaffold resistance are inherent to our platform's nature and limitations, the subsequent investigation will focus on understanding the impact of scaffold resistance on device characteristics, as detailed in the following sections.

### Effect of Scaffold Resistance on the Perovskite Solar Cell Performances

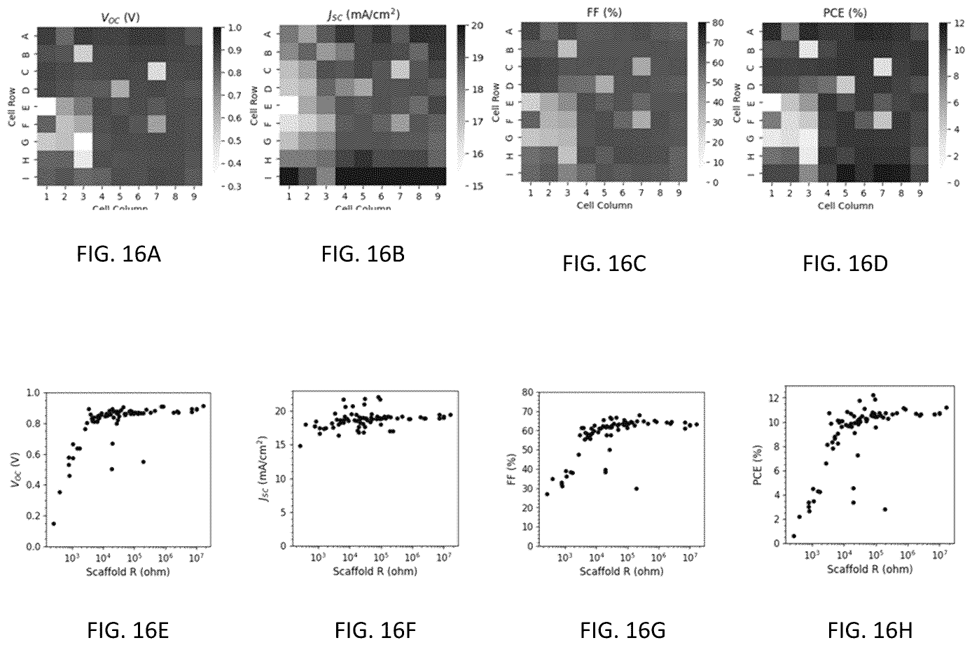

The influence of Scaffold Resistance (Scaffold R) on the performance of solar cells was investigated. Perovskite precursors, comprised of MAI (1.2 M), PbI2 (1.2 M), with AVAI (0.045 M) in GBL, were prepared and used to infiltrate a scaffold substrate comprising 81 cells. Subsequently, high-throughput J-V characterization was conducted. Photovoltaic parameters (V_{OC}, J_{SC}, FF, and PCE) were extracted from the J-V curves. FIGS. 16A to 16D illustrate the extracted parameters according to the physical position on the substrate. In particular, FIGS. 16A to 16D show the heatmaps of perovskite solar cell performance parameters based on the cell position for V_{OC} (FIG. 16A), J_{SC} (FIG. 16B), FF (FIG. 16C) and PCE (FIG. 16D). The heatmap represents the value of the corresponding parameters. Meanwhile, FIGS. 16E to 16H present the corresponding parameter plots with Scaffold R on the x-axis, that is, the respective plot of the parameter's value against Scaffold R.

In FIGS. 16E to 16H, it is observed that up to a certain value of Scaffold R, the parameters' value increases with Scaffold R. However, these parameters saturate when Scaffold R surpasses some threshold value (around 1×104 (Ohm)). A low Scaffold R implies a higher current leakage within the solar cells. The results suggest that, at a certain point, the photogenerated current can outweigh the leakage current, diminishing the impact of Scaffold R. This finding is pivotal for high-throughput characterization, emphasizing the protocol to exclude scaffold cells with Scaffold R below a specified threshold. This helps minimize measurement result bias contributed by factors before perovskite infiltration.

### Two-Probe Vs Four-Probe Measurement

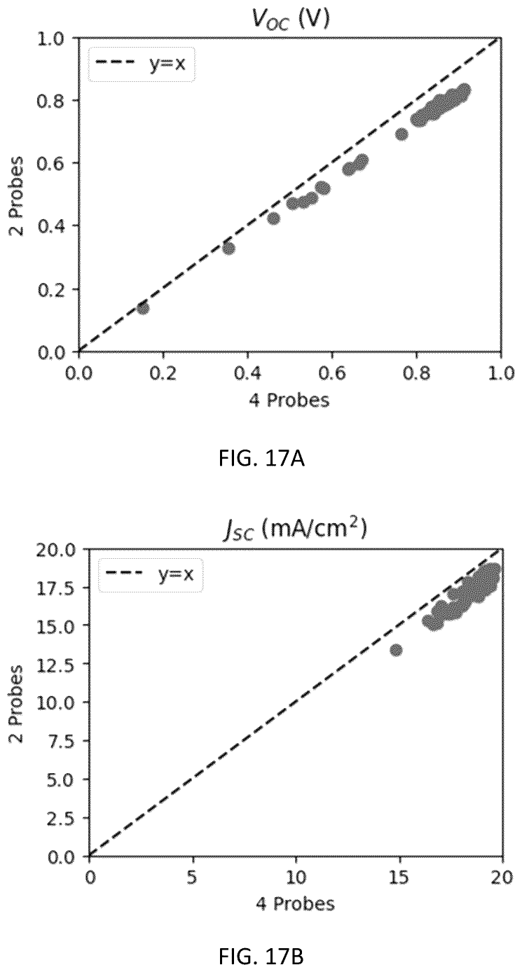

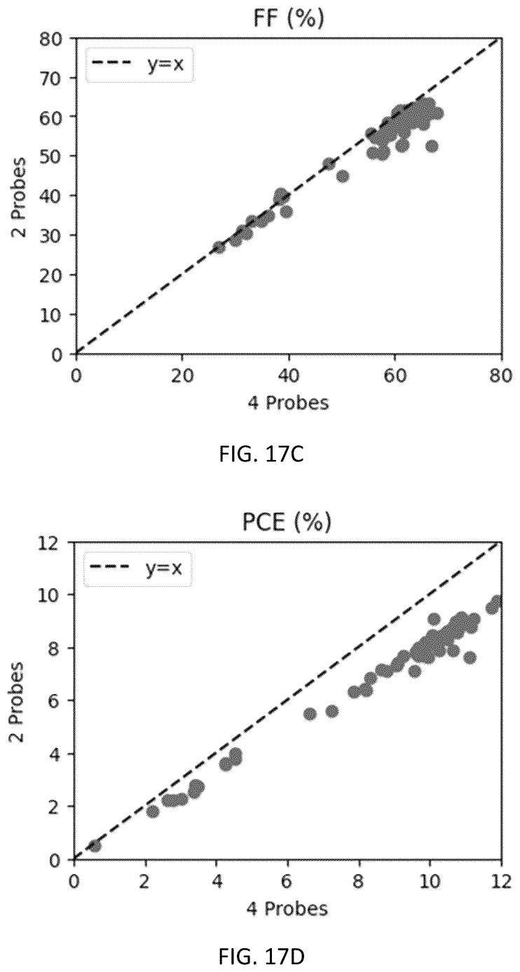

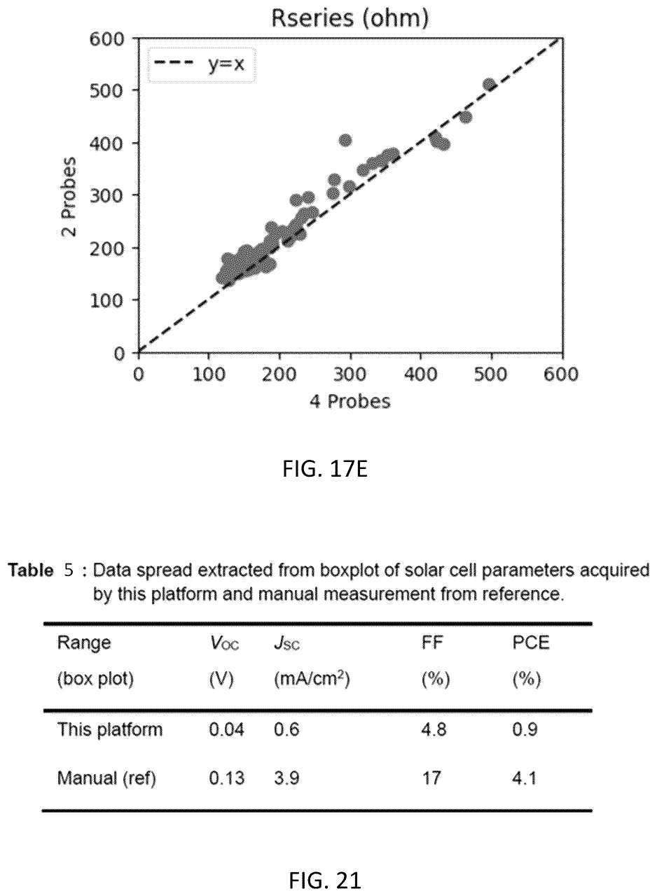

In the pursuit of reliable high-throughput J-V characterization, a comparison was made between two-probe and four-probe measurements. The four-probe measurement configuration comprises two probes configured to source and sink current through the perovskite solar cell (perovskite device) and two probes configured to sense a potential difference across the perovskite solar cell such that current and voltage paths are separated. In particular, in the four-probe measurement configuration, the Source Measurement Unit (SMU) employs separate pairs of current-carrying (“source”) and voltage-sensing (“sense”) leads to measure the current-voltage (J-V) characteristics of the perovskite solar cell. Two probes are used to source and sink current through the perovskite solar cell, while the other two probes are used exclusively to sense the potential difference across the active area. By separating the current and voltage paths, the four-probe setup effectively eliminates the influence of lead resistance, contact resistance, and wire resistance, which otherwise introduce voltage drops in conventional two-probe measurements. As a result, the voltage measured by the sensing probes reflects the true potential difference across the perovskite solar cell rather than a combination of intrinsic and parasitic resistances. This configuration provides more accurate determination of key photovoltaic parameters (e.g., such as open-circuit voltage (VOC), short-circuit current density (JSC), fill factor (FF), and power conversion efficiency (PCE)) especially in small-area or high-resistance devices where contact effects can be significant. To conduct this evaluation, a batch of cells with varying concentrations of 5-AVAI (5-Ammonium valeric acid iodide) was fabricated as a trial. These cells underwent J-V characterization under the AM1.5 at 1 sun spectrum. The key parameters such as V_{OC}, J_{SC}, FF, PCE, and Series Resistance were extracted from the J-V characteristic. A shadow mask with an area of 0.04 cm² was used. FIGS. 17A to 17E show the comparison between outcomes from four-probe (x-axis) and two-probe (y-axis) measurements. Dotted lines of y=x were incorporated as a reference to visually illustrate the disparities between the two measurement results. In particular, FIGS. 17A to 17E show two-probe vs four-probe measurement of (a) V_{OC}, (b) J_{SC}, (c) FF, (d) PCE, and (e) Rₛₑᵣᵢₑₛ, respectively, extracted from J-V characteristics. The dotted lines represent y=x as a reference to compare the disparity between four-probe and two-probe measurements.

The results in FIGS. 16A to 16H indicate that two-probe measurements consistently underestimate the values of V_{OC}, J_{SC}, FF, and PCE despite maintaining the Series Resistance (Rₛₑᵣᵢₑₛ) at a relatively constant level. Four-probe measurements by the Source Measurement Unit (SMU) would theoretically provide results closer to the ‘true’ values. The segregation of the source and sense in the SMU would theoretically minimize the impact of parasitic resistance arising from test leads, contact resistance, and wire resistance. The obtained results validate this hypothesis, showcasing the effectiveness of four-probe measurements in mitigating the influence of extraneous resistances and producing more accurate measurements. Consequently, the four-probe measurement method is chosen as the preferred protocol for high-throughput J-V measurements.

### Effect of Shadow Mask Size on the Solar Cell Performance Measurements

The impact of shadow mask size employed during J-V measurements on the solar cell performance was investigated. The primary purpose of using a shadow mask is to regulate the illuminated area on the solar cell, ensuring the homogeneity and the precision of the measurement.

A shadow mask made from a thin metal plate featuring 81 square holes was designed to precisely align with the positions of 81 cells on a substrate. Two shadow masks, with hole areas per cell of 0.09 cm² and 0.04 cm², were manufactured. To assess their efficacy, 81 solar cells, all sharing the same perovskite formulation (MAPbI₃ (1.2M) with AVAI (0.045 M)), were prepared on a single substrate. J-V characteristics were measured for cells without a mask, with a 0.09 cm² mask, and with a 0.04 cm² mask to compare the impact of mask size on data spread and accuracy. Ultimately, one of the masks was chosen as the standard protocol.

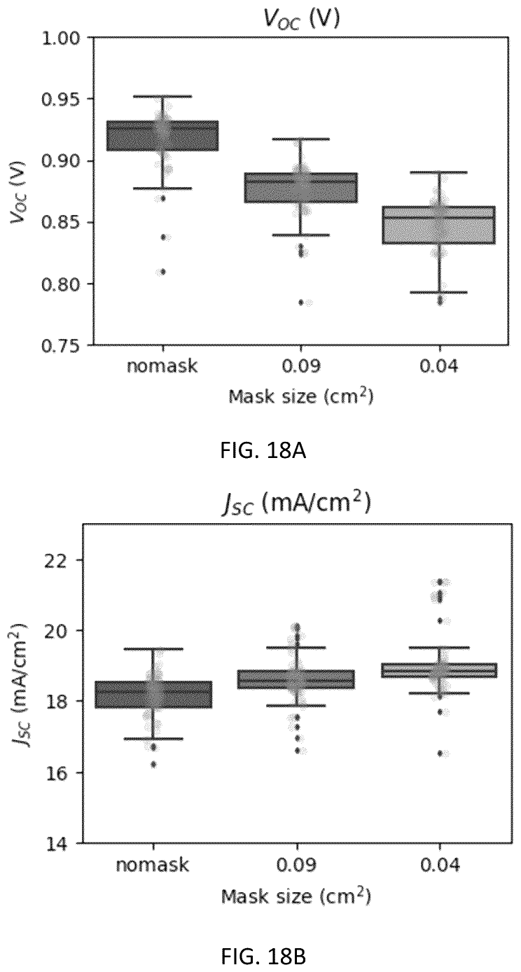

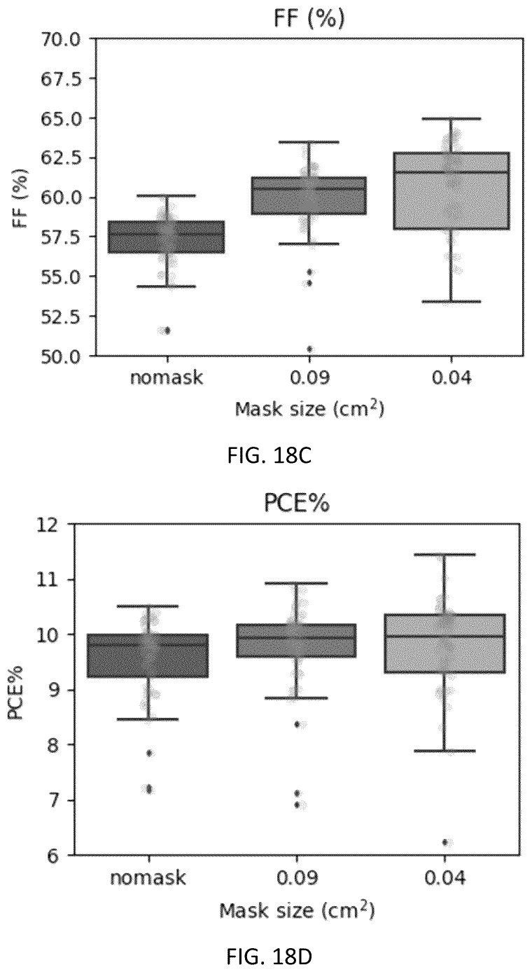

The boxplots of V_{OC}, J_{SC}, FF, and PCE of 81 cells extracted from the J-V characteristics are shown in FIGS. 18A to 18D. In particular, FIGS. 18A to 18D show solar cell performance parameters versus the mask size for (a) V_{OC}, (b) J_{SC}, (c) FF, and (d) PCE, respectively. The x-axis represents the measurement conditions without a mask (nomask), using 0.09 cm² mask, and 0.04 cm² mask. As seen from FIG. 18A, the V_{OC} decreases with the mask size. V_{OC} is proportional to ln(I₁/I₀−1), where I_{L} is photogenerated current and I₀ is saturated current dependent on carrier recombination. In solar cells, the photo-generated current is contributed only from the illuminated area, while carrier recombination can occur across the total area. Consequently, the disproportional carrier separation and recombination in smaller masks can be observed, manifested as a decrease in V_{OC}. By this means, the true V_{OC} value should be indicated by the measurement condition with a bigger or without a mask (nomask).

However, the spread of J_{SC} in ‘nomask’ condition is larger compared to smaller mask conditions (FIG. 18B). This discrepancy is likely attributed to the area variation resulting from the scaffold printing, leading to an inaccurate estimation of the current density.

In addition, from FF and PCE in FIGS. 18C and 18D, it becomes evident that the smallest mask (0.04 cm²) results in the largest data spread. Based on the above observations, a mask size of 0.09 cm² was selected as the standard protocol. It strikes a balance between avoiding substantial underestimation of V_{OC} and maintaining a narrow data spread across all parameters.

### Measurement Sequence Dependency

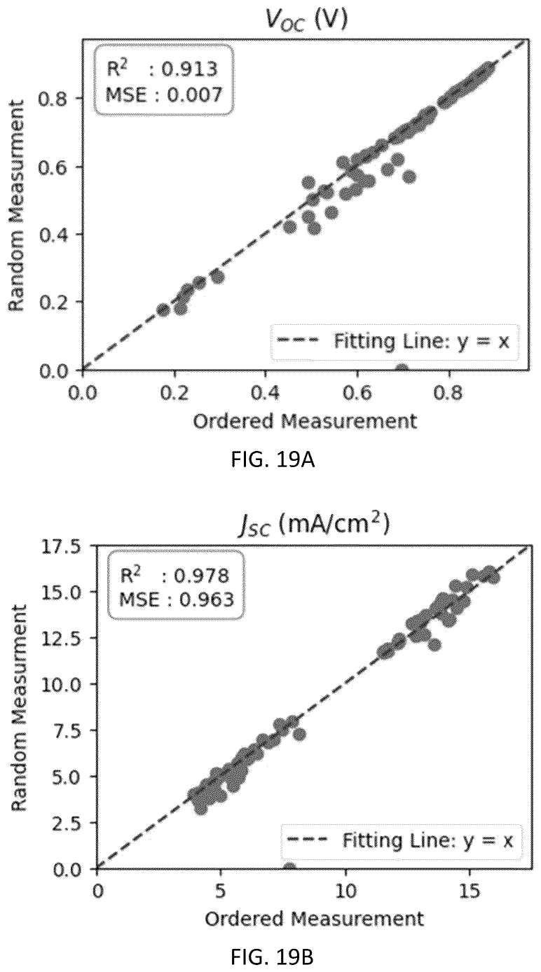

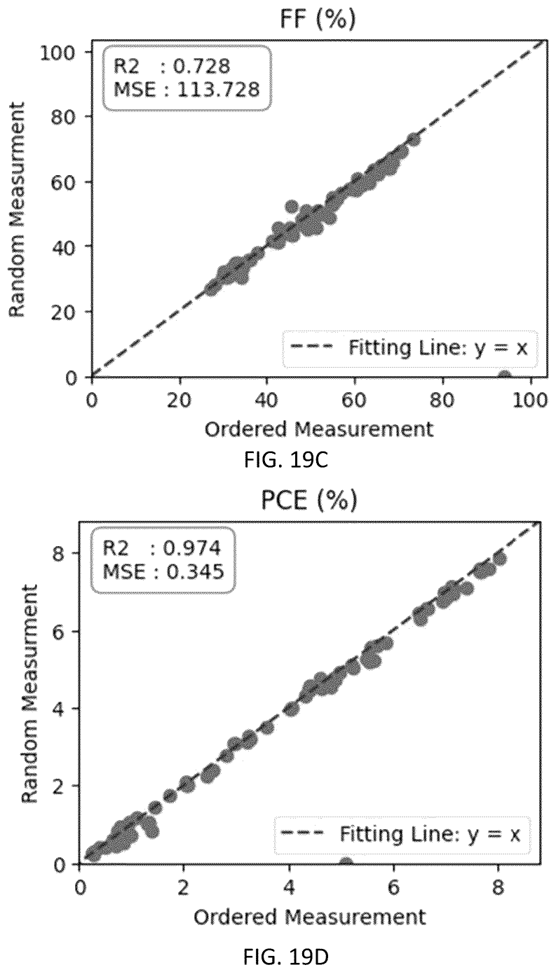

Measurement sequence dependency was examined by conducting ordered measurements (row by row, each in an orderly manner) and completely random sequences generated by computer. The results are presented in FIGS. 19A to 19D, which show the effect of measurement sequence on PV parameters. It shows that using established protocols so far, the effect of measurement sequence dependency can be neglected.

### Continuous Measurement Results Compared to Manual Measurement

Utilizing the protocols developed thus far, an automatic continuous measurement was carried out for cells with the same perovskite formula (MAPbI₃ (1.2M) with AVAI (0.045 M)). The high-throughput characterization platform was programmed to execute five loops of continuous J-V measurement on 81 perovskite solar cells to evaluate measurement reliability. The performance parameters extracted from the J-V characteristics against measurement numbers are presented in FIGS. 20A to 20D. In particular, FIGS. 20A to 20D show perovskite solar cell performance parameters acquired from the J-V curves in continuous measurement for (a) V_{OC}, (b) J_{SC}, (c) FF, and (d) PCE. The x-axis indicates the number of measurements. The range of the boxplot by manual measurement from reference is indicated on the right side of each graph. Note that the threshold value for the substrate resistance was set to 1×104 (Ohm). The average of the boxplot range was then compared with the data collected manually by our research group and summarized in Table 5 shown in FIG. 21. In particular, Table 5 shows the data spread extracted from boxplot of perovskite solar cell parameters acquired by this platform and manual measurement from reference. The results indicate that the high-throughput platform developed in this work exhibits an overall narrower data spread. It can narrow the data spread to approximately half compared to manual measurements by experienced experts. Implementing the high-throughput measurement platform has led to overall improvements in speed, accuracy, reduced human involvement time, and more importantly, decreased errors caused by human factors.

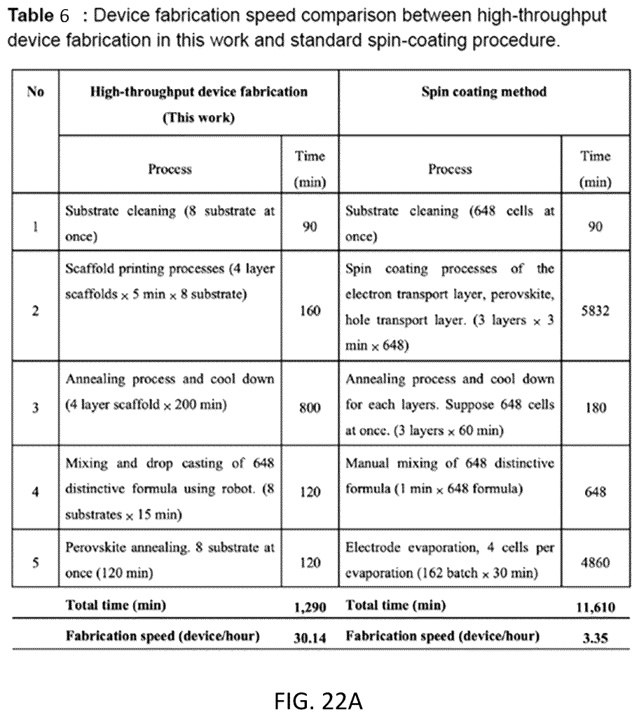

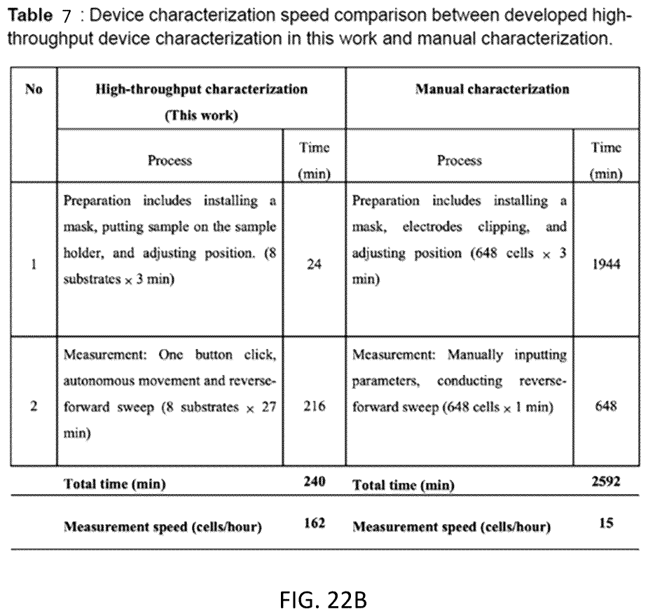

FIGS. 22A and 22B show Tables 6 and 7 presenting device fabrication speed and device characterization speed comparisons between the HT platform according to various example embodiments of the present invention and conventional manual work. In particular, Table 6 shows device fabrication speed comparison between the HT device fabrication according to various example embodiments of the present invention and conventional spin-coating procedure. Table 7 shows device characterization speed comparison between developed HT device characterization according to various example embodiments of the present invention and conventional manual characterization.

### Verification in Spin-Coated Devices

Spin-coated devices (FTO/c-TiO₂/m-TiO₂/Perovskite/Spiro-OMeTAD/Au) using the representative perovskite recipes according to various example embodiments were fabricated as a preliminary assessment for the transferability. No antisolvent was used during the spin-coating process due to the possibility of solubility issues with the additives. While these devices were produced with minimal optimization due to time and resource constraints, we observed meaningful consistencies with the trends reported in our mesoscopic devices, especially in stability.

20-XRD patterns of the devices were collected immediately after removal from the glovebox (0 h) and again after exposure to ambient air for 24 hours (24 h). The results in FIG. 11 align well with our observations in the mesoscopic architecture. Notably, the Champion device exhibits significantly reduced to no PbI₂ formation over time compared to the other samples, underscoring the synergistic effect of 5-AVAI and MACl.

In the freshly prepared samples, the presence of 5-AVAI promotes initial crystallization, as evidenced by sharper diffraction peaks and a diminished PbI₂ signal in Champion and AVAI only. MACl appears to help preserve the crystalline integrity, suggesting its role in enhancing long-term structural stability. These findings are consistent with the earlier results in mesoscopic devices according to various example embodiments of the present invention and further support the complementary functions of the two additives.

Accordingly, the method of optimizing material compositions for perovskite devices described herein according to various example embodiments of the present invention may be used partially or as a whole for material optimization toward a specific device application. An example use case for solution-processed perovskite solar cell optimization has been described and demonstrated. Perovskite source solution with different additives was mixed with different ratios to find the optimum composition for excellent solar cell performance and stability. As explained hereinbefore, the present invention is not limited to such a specific device application and other example use cases or practical applications include: perovskite memristor devices, perovskite-based neuromorphic devices, perovskite-based photoconductive devices, perovskite diodes (e.g., perovskite light emitting diodes (PeLEDs), perovskite photodetectors and so on, such as ionic liquid screening, lead-free perovskite solar cells optimization, perovskite material optimization for neuromorphic application, and so on.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

What is claimed is:

1. A method of optimizing material compositions for perovskite devices, the method comprising, for each iteration of a plurality of iterations:
providing a substrate having an array of multilayer device stacks formed thereon;
infiltrating, using an automated liquid handling robot, each multilayer device stack of the array of multilayer device stacks formed on the substrate associated with the iteration with a perovskite solution having a selected precursor composition from a set of different precursor compositions associated with the iteration to form a corresponding perovskite device, resulting in an array of perovskite devices associated with the iteration;
performing, using an automated electrical measurement probing robot, electrical characterization on each perovskite device of the array of perovskite devices for generating electrical characterization data associated with the array of perovskite devices associated with the iteration; and
predicting, using an artificial intelligence (AI) model, a new set of different precursor compositions for a next iteration or that the set of different precursor compositions associated with the iteration is optimal based on the electrical characterization data of the array of perovskite devices associated with the iteration.

2. The method according to claim 1, wherein, for each iteration of the plurality of iterations, each multilayer device stack of the array of multilayer device stacks is screen printed on the substrate.

3. The method according to claim 1, further comprising, for each iteration of the plurality of iterations: preparing, using the automated liquid handling robot and for each multilayer device stack of the array of multilayer device stacks associated with the iteration, the perovskite solution having the selected precursor composition for the multilayer device stack, including automated precursor mixing to obtain the perovskite solution having the selected precursor composition for infiltrating the multilayer device stack.

4. The method according to claim 1, wherein the AI model is an optimization model configured to predict the new set of different precursor compositions for the next iteration or that the set of different precursor compositions associated with the iteration is optimal based on the electrical characterization data of each perovskite device of the array of perovskite devices associated with the iteration with respect to one or more performance objectives for said each perovskite device based on different precursor compositions comprising different perovskite source precursors of different mixing ratios associated with the iteration.

5. The method according to claim 4, wherein:
the optimization model is a Bayesian Optimization (BO) model comprising one or more surrogate models and an acquisition function, and
for each iteration of the plurality of iterations, the method further comprises:
training, for each of the one or more surrogate models, the surrogate model based on the electrical characterization data of each perovskite device of the array of perovskite devices associated with the iteration with respect to a corresponding performance objective of the one or more performance objectives based on different precursor compositions comprising different perovskite source precursors of different mixing ratios associated with the iteration; and
predicting, using the acquisition function, the new set of different precursor compositions for the next iteration or that the set of different precursor compositions associated with the iteration is optimal based on the one or more surrogate models.

6. The method according to claim 1, wherein, for each iteration of the plurality of iterations, the array of multilayer device stacks formed on the substrate is an array of solar cells, and each solar cell of the array of solar cells comprises an electron transport layer, a spacer layer and a hole transport layer.

7. The method according to claim 6, wherein, for each iteration of the plurality of iterations, the electrical characterization data associated with the array of perovskite devices associated with the iteration comprises one or more of open-circuit voltage measurement data, short-circuit current density measurement data, fill factor measurement data and power conversion efficiency measurement data.

8. The method according to claim 6, wherein, for each iteration of the plurality of iterations, the electrical characterization on each perovskite device of the array of perovskite devices is performed using the automated electrical measurement probing robot according to a four-probe measurement configuration comprising two probes configured to source and sink current through the perovskite device and two probes configured to sense a potential difference across the perovskite device such that current and voltage paths are separated.

9. The method according to claim 6, wherein, for each iteration of the plurality of iterations, the electrical characterization on each perovskite device of the array of perovskite devices is performed further using an automated translation stage configured to move the substrate having the array of perovskite devices associated with the iteration supported thereon and a solar simulator configured to illuminate light that simulates sunlight onto the perovskite device.

10. The method according to claim 6, wherein:
the AI model is a multi-objective optimization model configured to predict the new set of different precursor compositions for the next iteration or that the set of different precursor compositions associated with the iteration is optimal based on the electrical characterization data of each perovskite device of the array of perovskite devices associated with the iteration with respect to one or more performance objectives for said each perovskite device based on different precursor compositions comprising different perovskite source precursors of different mixing ratios associated with the iteration, and
the one or more performance objectives comprise a peak power conversion efficiency objective defined to maximize a peak power conversion efficiency of perovskite devices and a power conversion efficiency change objective defined to minimize a power conversion efficiency change of perovskite devices.

11. A system for optimizing material compositions for perovskite devices, the system comprising:
an automated liquid handling robot configured to infiltrate each multilayer device stack of an array of multilayer device stacks formed on a substrate with a perovskite solution having a selected precursor composition from a set of different precursor compositions to form a corresponding perovskite device;
an automated electrical measurement probing robot configured to perform electrical characterization on each perovskite device of the array of perovskite devices for generating electrical characterization data associated with the array of perovskite devices; and
a computing system comprising:
at least one memory; and
at least one processor communicatively coupled to the at least one memory, the automated liquid handling robot and the automated electrical measurement probing robot and configured to, for each iteration of a plurality of iterations:
set the automated liquid handling robot with precursor composition set information on a set of different precursor compositions associated with the iteration for the automated liquid handling robot to infiltrate each multilayer device stack of an array of multilayer device stacks formed on a substrate associated with the iteration with a perovskite solution having a selected precursor composition from the set of different precursor compositions associated with the iteration to form a corresponding perovskite device, resulting in an array of perovskite devices associated with the iteration;
control the automated electrical measurement probing robot to perform electrical characterization on each perovskite device of the array of perovskite devices for generating electrical characterization data associated with the array of perovskite devices associated with the iteration; and
predict, using an artificial intelligence (AI) model, a new set of different precursor compositions for a next iteration or that the set of different precursor compositions associated with the iteration is optimal based on the electrical characterization data of the array of perovskite devices associated with the iteration.

12. The system according to claim 11, wherein, for each iteration of the plurality of iterations, each multilayer device stack of the array of multilayer device stacks is screen printed on the substrate.

13. The system according to claim 11, wherein, for each iteration of the plurality of iterations, the automated liquid handling robot is further configured to prepare, for each multilayer device stack of the array of multilayer device stacks associated with the iteration, the perovskite solution having the selected precursor composition for the multilayer device stack, including automated precursor mixing to obtain the perovskite solution having the selected precursor composition for infiltrating the multilayer device stack.

14. The system according to claim 11, wherein the AI model is an optimization model configured to predict the new set of different precursor compositions for the next iteration or that the set of different precursor compositions associated with the iteration is optimal based on the electrical characterization data of each perovskite device of the array of perovskite devices associated with the iteration with respect to one or more performance objectives for said each perovskite device based on different precursor compositions comprising different perovskite source precursors of different mixing ratios associated with the iteration.

15. The system according to claim 14, wherein:
the optimization model is a Bayesian Optimization (BO) model comprising one or more surrogate models and an acquisition function, and
for each iteration of the plurality of iterations, the at least one processor is further configured to:
train, for each of the one or more surrogate models, the surrogate model based on the electrical characterization data of each perovskite device of the array of perovskite devices associated with the iteration with respect to a corresponding performance objective of the one or more performance objectives based on different precursor compositions comprising different perovskite source precursors of different mixing ratios associated with the iteration; and
predict, using the acquisition function, the new set of different precursor compositions for the next iteration or that the set of different precursor compositions associated with the iteration is optimal based on the one or more surrogate models.

16. The system according to claim 11, wherein, for each iteration of the plurality of iterations, the array of multilayer device stacks formed on the substrate is an array of solar cells, and each solar cell of the array of solar cells comprises an electron transport layer, a spacer layer and a hole transport layer.

17. The system according to claim 16, wherein, for each iteration of the plurality of iterations, the electrical characterization data associated with the array of perovskite devices associated with the iteration cells comprises one or more of open-circuit voltage measurement data, short-circuit current density measurement data, fill factor measurement data and power conversion efficiency measurement data.

18. The system according to claim 16, wherein, for each iteration of the plurality of iterations, the electrical characterization on each perovskite device of the array of perovskite devices is performed using the automated probing robot according to a four-probe measurement configuration comprising two probes configured to source and sink current through the perovskite device and two probes configured to sense a potential difference across the perovskite device such that current and voltage paths are separated.

19. The system according to claim 16, further comprising:
an automated translation stage configured to, for each iteration of the plurality of iterations, move the substrate having the array of perovskite devices associated with the iteration supported thereon; and
a solar simulator configured to, for each iteration of the plurality of iterations, illuminate light that simulates sunlight onto the substrate associated with the iteration,
wherein the at least one processor is further configured to, for each iteration of the plurality of iterations:
control the automated translation stage to move the substrate having the array of perovskite devices associated with the iteration supported thereon for performing, for each perovskite device of the array of perovskite devices associated with the iteration, the electrical characterization on the perovskite device and for the solar simulator to illuminate the light onto the perovskite device.

20. The system according to claim 16, wherein:
the AI model is a multi-objective optimization model configured to predict the new set of different precursor compositions for the next iteration or that the set of different precursor compositions associated with the iteration is optimal based on the electrical characterization data of each perovskite device of the array of perovskite devices associated with the iteration with respect to one or more performance objectives for said each perovskite device based on different precursor compositions comprising different perovskite source precursors of different mixing ratios associated with the iteration, and
the one or more performance objectives comprise a peak power conversion efficiency objective defined to maximize a peak power conversion efficiency of perovskite devices and a power conversion efficiency change objective defined to minimize a power conversion efficiency change of perovskite devices.

21. The system according to claim 11, further comprising a perovskite device array forming apparatus configured to form, for each iteration of the plurality of iterations, the array of perovskite devices on the substrate associated with the iteration.
